(19) United States
(12) Patent Application Publication
(10) Pub. No.: US 2025/0114194 A1
(43) Pub. Date: Apr. 10, 2025

(54) UNIVERSAL STABILIZER FOR A DELIVERY SYSTEM

(71) Applicant: EDWARDS LIFESCIENCES CORPORATION, Irvine, CA (US)

(72) Inventors: Christopher Aguayo, Irvine, CA (US); Jesse Robert Edwards, Silverado, CA (US); Jonathan Joseph Panella, San Jose, CA (US); Brian Patrick Murphy, Irvine, CA (US); Omar Fawzi Azanki, Coto de Caza, CA (US); Harish Manickam Srinimukesh, Costa Mesa, CA (US); Alzuebeir Abdelbagi Elsheikh, Irvine, CA (US); Pablo Hernan Catania, Costa Mesa, CA (US); Paul Yen Tran, Anaheim, CA (US)

(21) Appl. No.: 18/981,330

(22) Filed: Dec. 13, 2024

Related U.S. Application Data
(63) Continuation of application No. PCT/US2023/024350, filed on Jun. 2, 2023.
(60) Provisional application No. 63/352,527, filed on Jun. 15, 2022.

(51) Int. Cl. A61F 2/24

(52) U.S. Cl. CPC A61F 2/2427

(57) ABSTRACT

A stabilizer for use with a delivery system is provided. The stabilizer receives a delivery system, such as a handle portion, and prevents unwanted movement of the delivery system during a medical procedure. The stabilizer desirably includes an elongate rail that is adapted to be secured to a base or table. A rail dock is adapted to releasably couple to the delivery system. The rail dock is slidably mounted along an upper surface of the rail and includes a brake assembly. The brake assembly preferably includes a toggle member with at least one push button, wherein actuation of the push button may release or lock the brake assembly. Components of the stabilizer are provided for allowing the stabilizer to be used with different delivery systems and the stabilizer is capable of being secured to different sized tables.

### CROSS-REFERENCE TO PRIORITY APPLICATIONS

This application is a continuation of International Application No. PCT/US2023/024350 filed Jun. 2, 2023, which claims the benefit of U.S. Provisional Application No. 63/352,527 filed Jun. 15, 2022, the entire content of which is hereby incorporated herein by reference.

### BACKGROUND

### Field

Certain embodiments disclosed herein relate generally to delivery systems for a prosthesis, and in some embodiments relate to a stabilizer and control systems for use with a delivery system for delivering a replacement heart valve, such as through a transseptal approach.

### Related Art

Human heart valves, which include the aortic, pulmonary, mitral and tricuspid valves, function essentially as one-way valves operating in synchronization with the pumping heart. The valves allow blood to flow downstream, but block blood from flowing upstream. Diseased heart valves exhibit impairments such as narrowing of the valve or regurgitation, which inhibit the valves' ability to control blood flow. Such impairments reduce the heart's blood-pumping efficiency and can be a debilitating and life-threatening condition. For example, valve insufficiency can lead to conditions such as heart hypertrophy and dilation of the ventricle. Thus, extensive efforts have been made to develop methods and apparatuses to repair or replace impaired heart valves.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open heart surgery. Replacement valves are being designed to be delivered through minimally invasive procedures and even percutaneous procedures. Such replacement valves often include a tissue-based valve body that is connected to an expandable frame that is then delivered to the native valve's annulus.

Development of prostheses including but not limited to replacement heart valves that can be compacted for delivery and then controllably expanded for controlled placement has proven to be particularly challenging. An additional challenge relates to the ability of such prostheses to be secured relative to intralumenal tissue, e.g., tissue within any body lumen or cavity, in an atraumatic manner.

Delivering a prosthesis to a desired location in the human body, for example delivering a replacement heart valve to the mitral valve, can also be challenging. Obtaining access to perform procedures in the heart or in other anatomical locations may require delivery of devices percutaneously through tortuous vasculature or through open or semi-open surgical procedures. The ability to control the location of a delivery system and the deployment of the prosthesis at the desired location can also be challenging.

### SUMMARY

According to a first universal stabilizer (which may be adapted for use with multiple different types of delivery systems, such as a transcatheter mitral valve replacement delivery system, a transcatheter tricuspid valve replacement delivery system, a transcatheter aortic valve replacement system, or other delivery system) of the disclosure, a rail extends along a longitudinal direction and includes a first end, a second end, an upper facing surface, a lower facing surface, and first and second sides extend between the first and second ends. A rail dock mounts on the upper facing surface of the rail. The rail dock includes first and second channel members spaced apart to receive the first and second sides of the rail therebetween. The first and second channel members include distal ends that overhang the lower facing surface of the rail and prevent removal of the rail dock from the rail in a vertical direction. A brake assembly can be actuated between a first configuration in which the rail dock translates along the rail and a second configuration in which the rail dock is prevented from translating along the rail.

According to another aspect of the first universal stabilizer, the rail dock includes a first support for a handle of a delivery system. According to another aspect of the first universal stabilizer, the rail dock includes a carriage oriented along the longitudinal direction, the first support mounted on the carriage and movable along the longitudinal direction relative to the rail dock. According to another aspect of the first universal stabilizer, the carriage is threadingly or threadedly mounted on a travel screw, travel screw connected with a twist knob for moving the carriage. According to another aspect of the first universal stabilizer, the first support includes a fixed member (e.g., clamp portion) and a movable member (e.g., clamp portion) pivotably coupled with the fixed member. The first support is actuatable between an open configuration and closed configuration. According to another aspect of the first universal stabilizer, a screw extends through an aperture in the movable member with a threaded end received within a threaded aperture in the fixed member. A knob is disposed outside of the movable member. According to another aspect of the first universal stabilizer, the first support is actuatable between the open configuration and the closed configuration by a rotation of the knob attached with the first support. The rotation of the knob can be less than 180°, or can be about 90° (e.g., between 80° and 100°). In some implementations, the knob can be rotated 1.5 turns to transition between the open configuration and the closed configuration.

According to another aspect of the first universal stabilizer, the brake assembly includes a toggle member with a first button on a first side of the brake assembly and a second button on a second side of the brake assembly. Pressing the first button shifts the brake assembly from the first configuration to the second configuration; pressing the second button shifts the brake assembly from the second configuration to the first configuration. According to another aspect of the first universal stabilizer, the brake assembly includes a brake member that engages and disengages from the upper facing surface of the rail. According to another aspect of the first universal stabilizer, the brake assembly includes a ramp connected with the toggle member. The ramp actuates the brake member to engage and disengage from the upper facing surface of the rail. In some configurations, disengagement of the brake member from the upper facing surface of the rail is accomplished by a spring force built in the brake member when the ramp is toggled to disengage the brake member. The brake member may be a springless brake without springs but having a spring force as a result of a design and materials of the brake member (e.g., a molded part with flexible extensions that can deform and return to an original state, thereby acting as springs without including springs). According to another aspect of the first universal stabilizer, the distal ends of the channel members are closer together than a width of the rail and fixed in position, such that the rail dock is loaded onto the rail by alignment of the first and second channels with the first and second sides at the first end of the rail and cannot be removed in a vertical direction. According to another aspect of the first universal stabilizer, a second support is coupled with the rail. According to another aspect of the first universal stabilizer, the second support is a hub nest configured to receive an introducer hub or sheath hub associated with a delivery system, the hub nest including a locking post that inserts within a receiving aperture in the upper facing surface of the rail and rotates to removably lock into place on the rail. According to another aspect of the first universal stabilizer, second support is a hub nest configured to receive an introducer hub or sheath hub associated with a delivery system, the hub nest including a locking post that inserts within a receiving aperture in the upper facing surface of the rail and slides in the longitudinal direction to removably lock into place on the rail. The hub nest may include a first embracing member (e.g., engagement or receiving member) and a second embracing member (e.g., engagement or receiving member) spaced apart to receive the first and second sides of the rail therebetween, each of the first and second channel members including a distal end that overhangs the lower facing surface of the rail and prevents removal of the hub nest from the rail in the vertical direction. According to another aspect of the first universal stabilizer, a second rail dock is mounted on the upper facing surface of the rail. The second rail dock actuates between a first configuration in which the second rail dock translates along the rail and a second configuration in which the second rail dock is prevented from translating along the rail.

According to another aspect of the first universal stabilizer, the first support includes a lock switch. The lock switch is rotatable between a locked position that holds the first support in the closed configuration, and an unlocked position in which the first support is allowed to move between the open and closed configurations. According to another aspect of the first universal stabilizer, the support includes an elastic strap with at first end secured to a first side of the support, a middle section extending over the handle, and a second side securable to a second side of the first support. According to another aspect of the first universal stabilizer, the support has a worm gear connected with a knob and a worm wheel connected with the handle, and rotation of the knob controls rotation of the handle about a longitudinal axis. According to another aspect of the first universal stabilizer, the rail dock includes a lockable support and the second rail dock includes a passive support.

According to a second universal stabilizer (which may be adapted for use with multiple different types of delivery systems, such as a transcatheter mitral valve replacement delivery system, a transcatheter tricuspid valve replacement delivery system, a transcatheter aortic valve replacement system, or other delivery system) of the disclosure, a rail extends along a longitudinal direction with a first end, a second end, an upper facing surface, a lower facing surface, and first and second sides extending between the first and second ends. A rail dock mounted on the upper facing surface of the rail. The rail dock has a first channel member on a first side of the rail dock aligned with the first side of the rail. A second channel member is on a second side of the rail dock. The second channel member is on a plate biased inwardly towards the second side of the rail. A button couples with the plate and pressing the button shifts the plate and second channel member away from the rail to permit the rail dock to translate along the rail. Releasing the button shifts the plate and the second channel member into the second side of the rail to prevent the rail dock from translating along the rail.

According to another aspect of the second universal stabilizer, the first and second channel members each include a projection with a distal end that overhangs the lower facing surface of the rail. According to another aspect of the second universal stabilizer, the rail dock includes a lockable support or a passive handle support. According to another aspect of the second universal stabilizer, a third channel member is biased into engagement with the either the first or second sides of the rail. According to another aspect of the second universal stabilizer, the first side of the rail includes a textured surface. According to another aspect of the second universal stabilizer, the second channel member is biased into engagement with the second side of the rail by a first spring force and the third channel member is biased into engagement with the first or second side of the rail by a second spring force, less than the first spring force. According to another aspect of the second universal stabilizer, a blocking member actuates between a secured position in which the rail dock is prevented from being removed from the rail in a vertical direction and an unsecured position in which the rail dock is permitted to be removed from the rail in the vertical direction. According to another aspect of the second universal stabilizer, the blocking member is a locking pin that, in the secure position and overhangs the lower facing surface of the rail.

According to a third universal stabilizer (which may be adapted for use with multiple different types of delivery systems, such as a transcatheter mitral valve replacement delivery system, a transcatheter tricuspid valve replacement delivery system, a transcatheter aortic valve replacement system, or other delivery system) of the disclosure, a rail has a first end, a second end, an upper facing surface, a lower facing surface, and first and second sides that extend between the first and second ends. A rail dock mounts on the upper facing surface of the rail. The rail dock has a first channel member along a first side of the rail dock. The first channel members includes a distal end that overhangs the lower facing surface on the first side of the rail. A second channel member is on a second side of the rail dock on a movable plate. A lever handle couples with the movable plate. The lever handle is movable between a fully locked configuration in which the rail dock is prevented from translating along the rail, a semi-locked configuration in which the rail dock is translating along the rail and not removable therefrom in a vertical direction, and a fully unlocked configuration in which the rail dock is removable from the rail in a vertical direction.

According to another aspect of the third universal stabilizer, the lever handle includes a base having a first side with a first extension width, a second side with a second extension width, and a third side with a third extension width. In the fully locked configuration, the first side of the base positions the plate and engages the second channel member with the rail. In the semi-locked configuration, the second side of the base positions the plate with the second channel member overhanging the lower facing surface of the rail. In the fully unlocked configuration, the third side of the base positions the plate with the second channel member disengaged from the rail.

According to a fourth universal stabilizer (which may be adapted for use with multiple different types of delivery systems, such as a transcatheter mitral valve replacement delivery system, a transcatheter tricuspid valve replacement delivery system, a transcatheter aortic valve replacement system, or other delivery system) of the disclosure, a rail extends along a longitudinal direction. The rail has a first end, a second end, an upper facing surface, a lower facing surface, first and second sides extending between the first and second ends. A rack on the rail extends in the longitudinal direction. A rail dock mounts on the upper facing surface of the rail. The rail dock has a pinion gear. The pinion gear is movable between a first position in which the pinion gear is engaged with the rack and a second position in which the pinion gear is not engaged with the rack. In the first position, the pinion gear is rotatable to adjust a position of the rail dock along the rail.

According to another aspect of the fourth universal stabilizer, the pinion gear is mounted on a spring loaded shaft attached with a rotation knob and depression of the knob moves the pinion gear between the first and second positions. According to another aspect of the fourth universal stabilizer, the pinion gear provides a torsional resistance to movement of the rail dock along the rail in the first position. According to another aspect of the fourth universal stabilizer, the rail dock includes first and second channel members spaced apart to receive the first and second sides of the rail therebetween. The first and second channel members include distal ends that overhang the lower facing surface of the rail and prevent removal of the rail dock from the rail in a vertical direction.

According to a surgical catheter system of the disclosure, a delivery system has a shaft assembly including a proximal end and a distal end. The delivery system may be, for example, a transcatheter mitral valve replacement delivery system, a transcatheter tricuspid valve replacement delivery system, a transcatheter aortic valve replacement system, or other delivery system. A handle assembly attaches with the proximal end of the shaft assembly. A lumen extends from the distal end of the shaft assembly to a proximal end of the handle. A stabilizer system for the delivery system, has a base and a handle support mounted on the base. The handle assembly is received within the handle support. A guidewire is disposed within the lumen. A proximal section of the guidewire extending proximally from the handle assembly. A guidewire management system for controlling movement of the guidewire relative to the handle assembly has an actuator to advance and retract along an axis aligned with the lumen. An engagement clamp releasably secures the guidewire relative to the actuator. The proximal section of the guidewire is received within the engagement clamp. A support for the actuator couples with the base. A user interface receives a user input. A controller moves the actuator to selectably advance and retract the guidewire along the axis based on the user input.

A guidewire management system of the disclosure includes a guidewire. An engagement clamp releasably secures about the guidewire. An actuator advances and retracts the engagement clamp along an axis. A support attached with the actuator. A user interface generates a user input signal. A controller moves the actuator to selectably advance and retract the guidewire along the axis based on the user input signal.

According to another aspect of the disclosure, the guidewire management system includes a stabilizer system for supporting a handle assembly of the delivery system on a base. The support couples with the base and extends proximally relative to a distal end of the stabilizer system. According to another aspect of the guidewire management system, the axis is aligned with a lumen of the handle assembly of the delivery system, the guidewire disposed within the lumen. According to another aspect of the guidewire management system, the engagement clamp is actuatable between a locked configuration and an unlocked configuration. According to another aspect of the guidewire management system, the user interface includes a lock button and an unlock button and the controller actuates the engagement clamp between the locked and unlocked configurations based on the user input signal. According to another aspect of the guidewire management system, the engagement clamp is manually actuatable. According to another aspect of the guidewire management system, the engagement clamp includes a passive fixing groove for securing the guidewire. According to another aspect of the guidewire management system, the user interface includes an advance button and a retract button. According to another aspect of the guidewire management system, the user interface includes a coarse advance button, coarse retract button, a fine advance button, and a fine retract button. According to another aspect of the guidewire management system, the actuator includes a servo controller that measures a position of the guidewire along the axis relative to an initial position, the servo controller provides positional feedback to the controller. According to another aspect of the guidewire management system, a load sensor that measures a force applied to the guidewire by the actuator, the load sensor provides force feedback to the controller. According to another aspect of the guidewire management system, a wireless interface transmits the user input signal from the user interface to the controller mounted on the support. According to another aspect of the guidewire management system, the controller generates a motor control signal based on the user input signal, and the actuator receives the motor control signal and to advance or retract the guidewire along the axis based on the motor control signal. According to another aspect of the guidewire management system, the motor control signal is further based on a position of the guidewire or a force exerted on the guidewire by the actuator.

According to a delivery system for delivering an expandable implant to a body location of the disclosure, an outer sheath assembly has an outer shaft with an outer lumen and a proximal end and a distal end. The outer sheath assembly has an implant retention area to retain the expandable implant in a compressed configuration. A rail assembly is located within the outer lumen, the rail assembly has a rail shaft having a rail lumen and a proximal end and a distal end. The rail assembly has one or more pull wires attached on an inner surface of the rail shaft that provide an axial force on the rail shaft to steer the rail assembly. An inner assembly located within the outer lumen has an inner shaft having an inner lumen and a proximal end and a distal end. The inner assembly has an inner retention member that is releasably attached to the expandable implant. The outer sheath assembly and the inner assembly move together distally relative to the rail assembly while the expandable implant remains in the compressed configuration. The outer sheath assembly retracts proximally relative to the inner assembly in order to at least partially expand the expandable implant from the compressed configuration. A mid shaft assembly within the outer lumen has a mid shaft having a middle lumen and a proximal end and a distal end. The mid shaft assembly has an outer retention member to radially restrain at least a portion of the expandable implant. A nose cone assembly located within the inner lumen, the nose cone assembly has a nose cone shaft having a guide wire lumen, a proximal end, a distal end, and a nose cone on the distal end. The mid shaft assembly and the nose cone assembly move together distally with the outer sheath assembly and the inner assembly relative to the rail assembly while the expandable implant remains in the compressed configuration. The mid shaft assembly retracts proximally relative to the inner assembly in order to at least partially expand the expandable implant from the compressed configuration. The nose cone assembly includes a force sensor. A handle has a haptic feedback system coupled with the force sensor and alerts a user if forces in excess of a predetermined threshold are detected.

According to a handle for a delivery system of the disclosure, a rail housing has a first rotatable actuator coupled with a first pull wire and provides an axial force on the first pull wire. A first encoder measures a position of the first rotatable actuator. A second rotatable actuator has a second pull wire and to provide an axial force on the second pull wire. A second encoder measures a position of the second rotatable actuator. A delivery housing has a third rotatably actuator coupled with an outer sheath assembly that move the outer sheath assembly distally relative to the delivery housing. A third encoder measures a position of the third rotatable actuator. A fourth rotatable actuator couples with a mid shaft assembly and retracts proximally the mid shaft assembly relative to the delivery housing. A fourth encoder measures a position of the fourth rotatable actuator. A fifth rotatable actuator moves the delivery housing relative to the rail housing. A fifth encoder measures a position of the fifth rotatable actuator. The delivery system may include, for example, a transcatheter mitral valve replacement delivery system, a transcatheter tricuspid valve replacement delivery system, a transcatheter aortic valve replacement system, or other delivery system.

According to another aspect, the handle includes a processor to receive a signal from each of the first, second, third, fourth, and fifth encoders and output a positional status of each of the first, second, third, fourth, and fifth rotatable actuators. According to another aspect, the handle has or is connected with a user interface to display the positional status of each of the first, second, third, fourth, and fifth rotatable actuators. One or more of the encoders may be mechanical encoders with detents or other clicking features to measure rotational position and provide haptic and/or audible feedback.

According to another aspect, a universal stabilizer includes a motorized rail system. A support is mounted on the motorized rail system. The support receives a handle of a delivery system. A control system moves the support between first and second ends of the motorized rail system. According to another aspect, a user interface receives a user input signal and generates a motor control signal for the control system based on the user input signal. According to another aspect, the motorized rail system includes a threaded shaft coupled with a motor and the support is mounted on a threaded carriage engaged with the threaded shaft.

In accordance with one aspect, a universal stabilizer adapted or configured for use with multiple different delivery systems (which may include, for example, one or more transcatheter mitral valve replacement delivery systems, one or more transcatheter tricuspid valve replacement delivery systems, one or more transcatheter aortic valve replacement systems, or one or more other delivery systems) is disclosed. The stabilizer includes a rail extending along a longitudinal direction and having a first end, a second end, an upper facing surface, a lower facing surface, and first and second sides extending between the first and second ends; and a rail dock mounted on the upper facing surface of the rail. The rail dock includes first and second channel members spaced apart to receive the first and second sides of the rail therebetween, the first and second channel members including distal ends that overhang the lower facing surface of the rail and prevent removal of the rail dock from the rail in a vertical direction. The rail dock further includes a brake assembly configured to be actuated between a first configuration in which the rail dock is configured to translate along the rail and a second configuration in which the rail dock is prevented from translating along the rail. The brake assembly includes a toggle member with at least one push button configured to cause the brake assembly to transition between the first configuration and the second configuration.

The toggle member may include a first button on a first side of the brake assembly and a second button on a second side of the brake assembly, wherein pressing the first button actuates (e.g., shifts, transitions) the brake assembly from the first configuration to the second configuration and pressing the second button actuates the brake assembly from the second configuration to the first configuration.

In some configurations, the stabilizer is configured to operate in conjunction with a guidewire management system. The stabilizer may be operably coupled to the rail or a base to which the rail is attached, wherein the guidewire management system is configured to support a guidewire over which a delivery system is configured to be advanced. The guidewire management system may be configured to cause movement of the guidewire (e.g., advancement, retraction, and or rotation) with respect to the delivery system.

A surgical system may include any of the stabilizers disclosed herein in combination with any one or more of the delivery systems disclosed herein and/or any of the guidewire management systems disclosed herein. For example, the surgical system may include a base or platform to which the rail of the stabilizer is configured to be attached. The delivery system may include a handle with which a support of the stabilizer is configured to be engaged. The delivery system may be configured to deliver a prosthetic heart valve to replace a native heart valve (e.g., a mitral valve, aortic valve, pulmonary valve or tricuspid valve).

The foregoing summary is illustrative only and is not intended to be limiting. Other aspects, features, and advantages of the systems, devices, and methods and/or other subject matter described in this application will become apparent in the teachings set forth below. The summary is provided to introduce a selection of some of the concepts of this disclosure. The summary is not intended to identify key or essential features of any subject matter described herein.

### BRIEF DESCRIPTION OF THE DRAWINGS

Various examples are depicted in the accompanying drawings for illustrative purposes, and should in no way be interpreted as limiting the scope of the examples. Various features of different disclosed examples can be combined to form additional examples, which are part of this disclosure.

FIG. 1A is a perspective view of a universal stabilizer coupled to a delivery system;

FIG. 1B is a perspective view of an alternative embodiment of a universal stabilizer coupled to a delivery system;

FIG. 1C is a perspective view of the universal stabilizer of FIG. 1A including two supports for the delivery system;

FIG. 1D is a side view of the universal stabilizer of FIG. 1C;

FIG. 1E is a perspective view of the universal stabilizer of FIG. 1B including two supports for the delivery system;

FIG. 2 is a perspective view of the first support of FIGS. 1A-1E mounted on a rail dock;

FIG. 3 is a perspective view of the rail dock of FIG. 2 with a portion of the housing removed;

FIG. 4 is a side view of the rail dock of FIG. 2;

FIG. 5A is a front view of the rail dock of FIG. 2;

FIG. 5B is a sectional view taken along the line 5B-5B in FIG. 4;

FIG. 6 is an exploded view of components of the first support of FIG. 3;

FIG. 7 is a perspective view of a brake assembly of the rail dock of FIG. 2 with the housing removed for clarity;

FIG. 8 is an exploded view of the brake assembly of FIG. 7;

FIG. 8A is a perspective view of an alternative embodiment of a brake assembly;

FIG. 8B is a perspective view of an alternative embodiment of a bottom plate of the brake assembly of FIG. 8 with a detent formed therein.

FIG. 9 is a cross-sectional view taken along the line 9-9 in FIG. 4;

FIG. 10 is a cross-sectional view taken along the line 10-10 in FIG. 4;

FIG. 11 is a perspective view of a second support of the universal stabilizer of FIG. 1;

FIG. 11A is a perspective view of an alternative embodiment of the hub nest of the second support in FIG. 11.

FIG. 11B is cross-sectional view of the hub nest of FIG. 11A taken from line 11B-11B.

FIG. 12A is a bottom perspective view of the second support of FIG. 11;

FIG. 12B shows assembly of the second support with the rail of the universal stabilizer;

FIG. 12C is a top view of the second support in an unlocked position on the rail;

FIG. 12D is a top view of the second support in a locked position on the rail;

FIG. 12E is a partial cross-sectional view showing the second support locked within the rail;

FIG. 12F shows a hub nest locked in the second support;

FIG. 13 is a perspective view of another embodiment of a stabilizer including three supports for a delivery system mounted on a rail;

FIG. 14 is a perspective view of a handle for a delivery system supported by the stabilizer of FIG. 13;

FIG. 15A is a perspective view of a first support and first rail dock of the stabilizer system of FIG. 13;

FIG. 15B is an exploded view of the first support of FIG. 15A;

FIG. 16A is a perspective view of a second support and second rail dock of the stabilizer system of FIG. 13;

FIG. 16B is an exploded view of the second support of FIG. 16A;

FIG. 17A is a perspective view of a third support and first rail dock of the stabilizer system of FIG. 13;

FIG. 17B is an exploded view of the third support of FIG. 17A;

FIG. 18A is a perspective view of another embodiment of a rail dock;

FIG. 18B shows a detailed view of the rail dock of FIG. 18A;

FIG. 18C shows a bottom view of the rail dock of FIG. 18A including two spring loaded channel members for gripping a rail;

FIG. 19A is a side view of another embodiment of a support of a stabilizer with a locking switch;

FIG. 19B is a perspective view of the support of FIG. 19A in an opened configuration;

FIG. 19C is a perspective view of the support of FIG. 19A in a locked configuration;

FIG. 20A is a perspective view of another embodiment of a support that can be used with a stabilizer;

FIG. 20B shows the support of FIG. 20A in an opened configuration;

FIG. 21 is a perspective view of another embodiment of a support with an elastic strap that can be used with a stabilizer for a delivery system;

FIG. 22 is a perspective view of another embodiment of a support with a rotational feature that can be used with a stabilizer for a delivery system;

FIG. 23A is a perspective view of another embodiment of a rail dock including a lever in a locked position;

FIG. 23B is a perspective view of the rail dock of FIG. 23A with the lever in a semi-locked position;

FIG. 23C is a perspective view of the rail dock of FIG. 23A with the lever in a fully unlocked position;

FIG. 23D is a plan view of a base of the rail dock of FIGS. 23A-23C;

FIG. 24A is a perspective view of another embodiment of a rail dock including a lever in a locked position;

FIGS. 24B-C are perspective views of the rail dock of FIG. 23A with the lever in a semi-locked position;

FIG. 24D is a perspective view of the rail dock of FIG. 23A with the lever in a fully unlocked position;

FIG. 24E is a plan view of a base of the rail dock of FIGS. 24A-24C;

FIG. 25A is a perspective view of another embodiment of a rail dock including a push button for actuating the rail dock between a locked configuration and an unlocked configuration;

FIG. 25B is a perspective view of a locking mechanism for preventing removal of the rail dock from the rail in a vertical direction;

FIG. 25C is a perspective view of the locking mechanism of FIG. 25B rotating into a locked position;

FIG. 26 is a perspective view of another embodiment of a stabilizer including a rail and a rail dock;

FIG. 27A is a plan view of the rail dock including a pinion gear disengaged from a rack on the rail;

FIG. 27B is a plan view of the pinion gear of the rail dock engaged with the rack;

FIG. 28 is a perspective view of a clamp integrated with a bottom of the rail of the stabilizer of FIG. 26;

FIG. 29 is a perspective view of a stabilizer including a guidewire management system;

FIG. 30 is a perspective view of a user interface of the guidewire management system integrated on a handle of the delivery system;

FIG. 31 is a perspective view of a support actuator of the guidewire management system;

FIG. 32 is a perspective view of a delivery system including a handle with a haptic feedback device;

FIG. 33 is a cross-sectional view of a nose cone assembly of the delivery system of FIG. 32 including a force sensor;

FIG. 34 is a perspective view of a handle of a delivery system including a plurality of knobs for articulating the delivery system;

FIG. 35 is a cross-sectional view of the handle with a plurality of encoders for measuring positions of a plurality of control knobs of the handle of the delivery system;

FIG. 36 is a perspective view of a universal stabilizer for a delivery system including a support for a handle that is movable along a motorized rail system.

### DETAILED DESCRIPTION

The various features and advantages of the systems, devices, and methods of the technology described herein will become more fully apparent from the following description of the examples illustrated in the figures. These examples are intended to illustrate the principles of this disclosure, and this disclosure should not be limited to merely the illustrated examples. The features of the illustrated examples can be modified, combined, removed, and/or substituted as will be apparent to those of ordinary skill in the art upon consideration of the principles disclosed herein.

The present specification and drawings provide aspects and features of the disclosure in the context of several embodiments of replacement heart valves, delivery systems and methods. The disclosed delivery systems are configured for use in the vasculature of a patient, such as for replacement of natural heart valves in a patient. These embodiments may be discussed in connection with replacing specific valves such as the patient's aortic, tricuspid, pulmonary, or mitral valve. However, it is to be understood that the features and concepts discussed herein can be applied to products other than heart valve implants. For example, the controlled positioning, deployment, and securing features described herein can be applied to medical implants, for example other types of expandable prostheses, for use elsewhere in the body, such as within an artery, a vein, or other body cavities or locations. In addition, particular features of a valve, delivery system, etc. should not be taken as limiting, and features of any one embodiment discussed herein can be combined with features of other embodiments as desired and when appropriate. While certain of the embodiments described herein are described in connection with a transfemoral (or transseptal) delivery approach, it should be understood that these embodiments can be used for other delivery approaches, such as, for example, transapical or transjugular approaches. Moreover, it should be understood that certain of the features described in connection with some embodiments can be incorporated with other embodiments, including those which are described in connection with different delivery approaches.

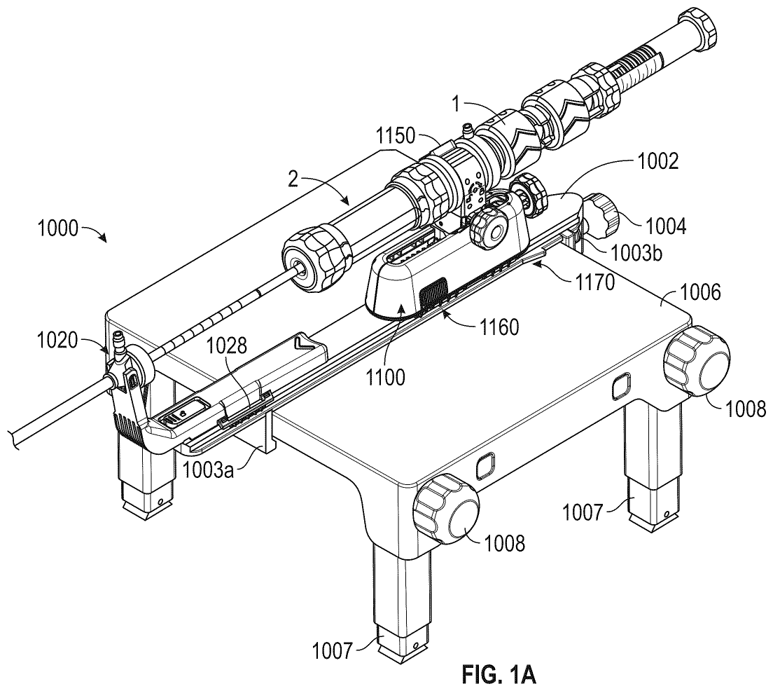

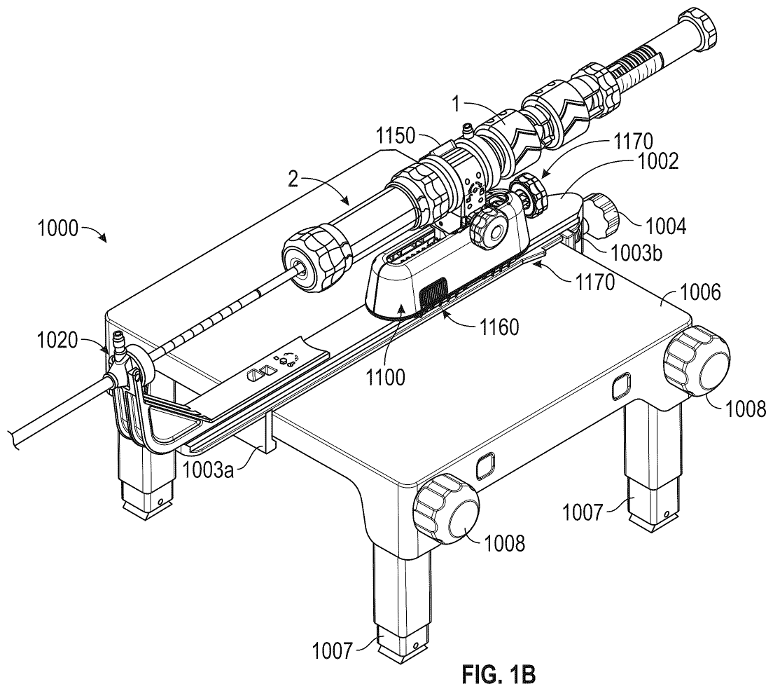

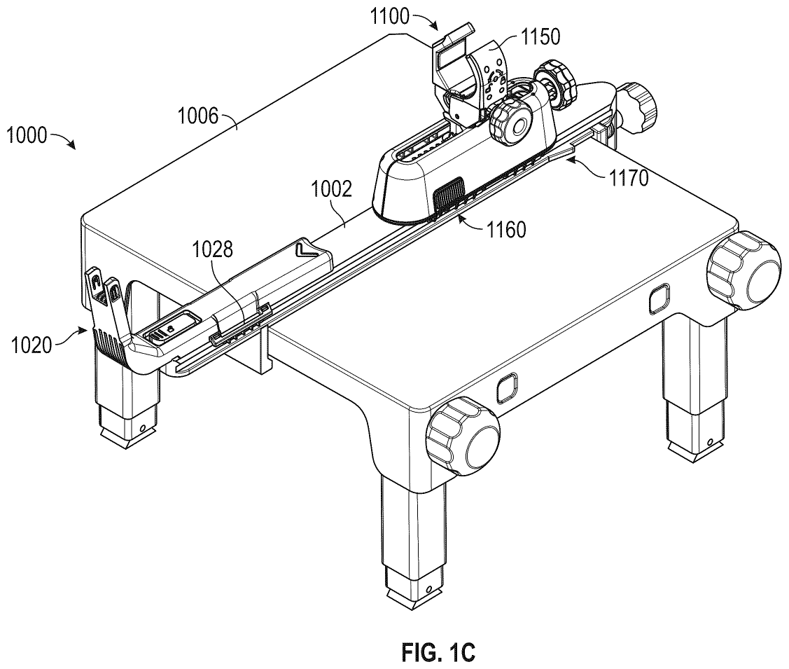

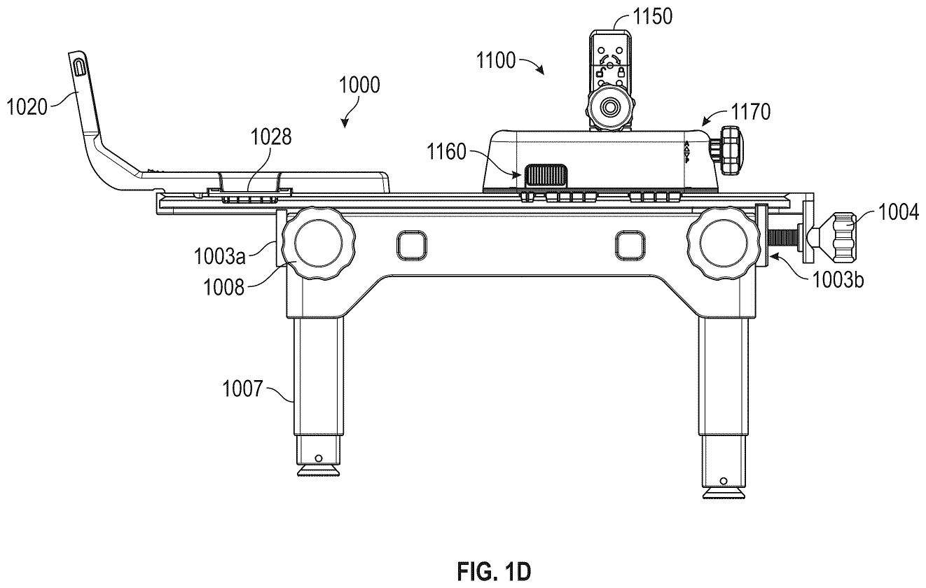

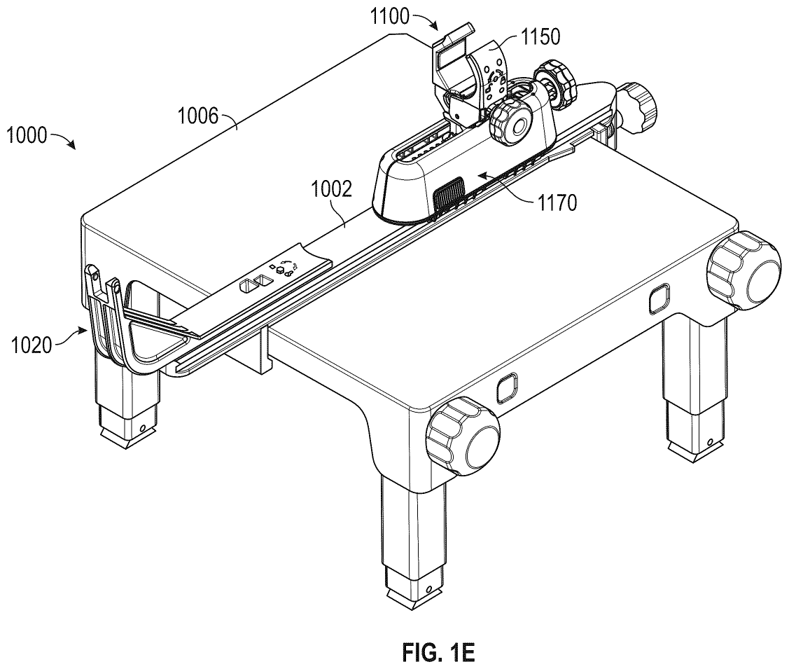

Embodiments of stabilizers 1000, shown in FIGS. 1A-E, can hold embodiments of a delivery system 2 having a handle 1 in proper positioning during use. FIG. 1A illustrates an embodiment of a stabilizer 1000, e.g., a universal stabilizer, engaged with a delivery system 2 for delivering a prosthesis to a body location. FIG. 1B shows an alternative embodiment of the stabilizer 1000 in FIG. 1A, holding a delivery system 2. FIGS. 1C and 1D are views of the stabilizer 1000 of FIG. 1A without the delivery system 2. FIG. 1E is a perspective view of the stabilizer 1000 in FIG. 1B without the delivery system 2.

Generally, the stabilizer 1000 can be used to hold a delivery system 2 in place, for example, above a patient's leg or on an operating table, though the particular position is not limiting. The stabilizer 1000 enables the delivery system 2 to remain stable during the procedure. In some embodiments, the stabilizer 1000 can be used to torque (rotate), advance, and/or retract components (independently or simultaneously) of the delivery system 2 in a controlled manner. Examples of delivery systems 2 that may be held with the stabilizer 1 are described in detail in U.S. Pat. Pub. No. 2019/0008640, the entirety of which is hereby incorporated by reference. Examples of other stabilizers are disclosed in U.S. Pat. Pub. No. 2020/0108225, the entirety of which is hereby incorporated by reference. The disclosed stabilizer 1000 can be advantageous for a transseptal (e.g., transfemoral) approach for delivering a replacement heart valve by allowing for fine motor control of a delivery system within the stabilizer. However, the embodiments of the stabilizers disclosed herein can be used for other approaches and other procedures as well, such as transapical approaches, and are not limited to replacement heart valves. The stabilizers 1000 may be universal in that they can be coupled to, or used with, any of a variety of different delivery systems (e.g., delivery systems for delivering replacement aortic valves, systems for delivering replacement mitral valves, systems for delivering replacement tricuspid valves, systems for delivering replacement pulmonary valves, or other delivery systems.

As shown in FIGS. 1A-E, the stabilizer 1000 can include a base or platform 1006. The base 1006 can be a stool, a table or other flat surface. In some embodiments, the base 1006 may not be used. In some embodiments, the base 1006, can be, for example, placed over a patient's leg in order to help support the stabilizer 1000. The base 1006 can be sized to properly interact with the stabilizer 1000. In some embodiments, the base can include a generally flat upper surface with a number of legs 1007 extending downwards from that surface. Thus, a patient may extend his/her legs through gaps between adjacent legs as needed. In some embodiments, the legs 1007 can be adjustable in order to vary the height of the upper surface. In some embodiments, a stabilizer can further include a plate or other hard surface which can be placed under a patient for providing a stable surface for the base 1006 to be placed on. Each of the legs 1007 can be independently or semi-independently extended or retracted (operable by a knob 1008) to adjust an angle of the top surface of the base 1006 and a rail 1002.

In some embodiments, the stabilizer 1000 can be a universal stabilizer system. This system can be easily adaptable for different sized bases and delivery systems. The stabilizer can utilize the universally attachable rail 1002, which may allow more flexibility and adaptability. The rail 1002 can include a distal end and a proximal end. In general, the proximal end is toward the user of the stabilizer 1000 (e.g., a clinician or healthcare professional) and/or the delivery system 2 that is coupled to the stabilizer 1000, and the distal end is away from the user. The rail 1002 can extend along a longitudinal direction. The rail 1002 can include one or more clamps. The rail 1002 may be attached directly to the base 1006, such as at the upper surface. The rail 1002 can be attached by clamps with the base 1006. The rail 1002 can include a moveable clamp 1003b (operable by a knob 1004) and a stationary clamp 1003a spaced longitudinally apart. In some embodiments, both clamps may be moveable. As discussed in detail below, the moveable clamp can be locked at a desired position on the rail, thus allowing the rail to be attached to different sized surfaces. The rail and the clamps can both be reusable and sterilizable and can be used for multiple different types of delivery or repair systems (e.g., replacement heart valve delivery systems or heart valve repair systems). In accordance with several implementations, a single knob 1004 (e.g., one and only one knob) may be used for clamping and unclamping the rail 1002 to the base 1006.

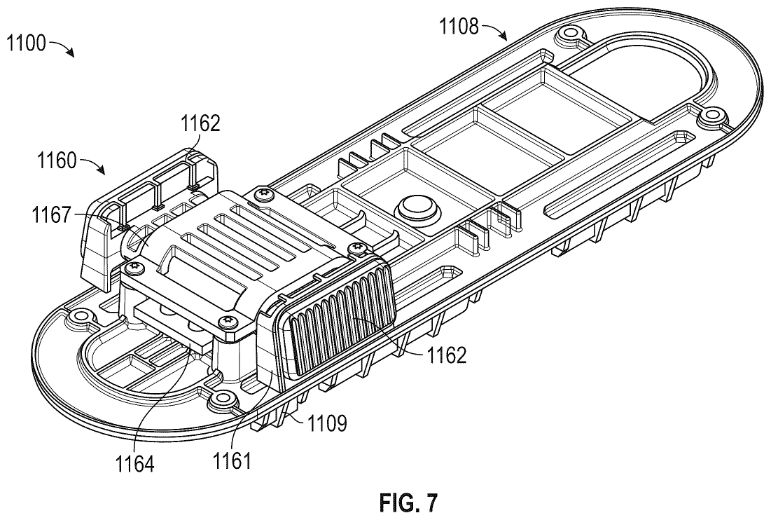

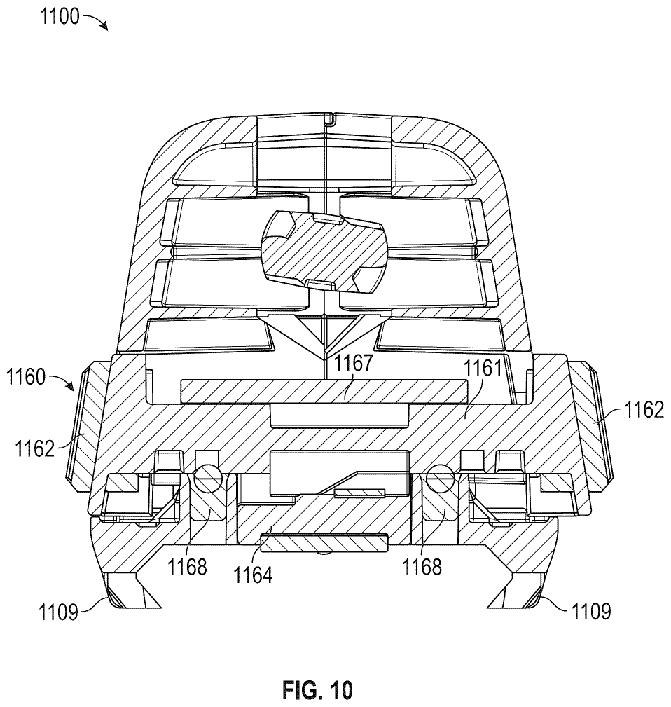

The stabilizer 1000 can further include a rail dock 1100 and a hub nest 1020, both coupled with the rail 1002. The rail dock 1100 can include a support 1150, a carriage assembly 1170, and/or a braking system 1160. In some embodiments, multiple rail docks may be used along the rail 1002. In some embodiments, the rail dock 1100 can have an adjustable upper surface for adjusting angles. The rail dock 1100 can be mountable on an upper surface of the rail 1002 and moveable along the longitudinal direction. The rail dock 1100 can be locked in place along the rail 1002 by the braking system 1160. As shown in FIGS. 7-10, the rail dock 1100 may have a bottom plate 1108 with channel members or protrusions 1109 (and distal ends 1109a) that at least partially wrap around a top surface of the rail 1002 for allowing the rail dock 1100 to be secured with the rail 1002, while preventing removal in a vertical direction. The protrusions 1109 can be spaced apart to receive a width of the rail therebetween. The rail dock 1100 can be mounted over the rail 1002 at one or both of the proximal and distal ends.

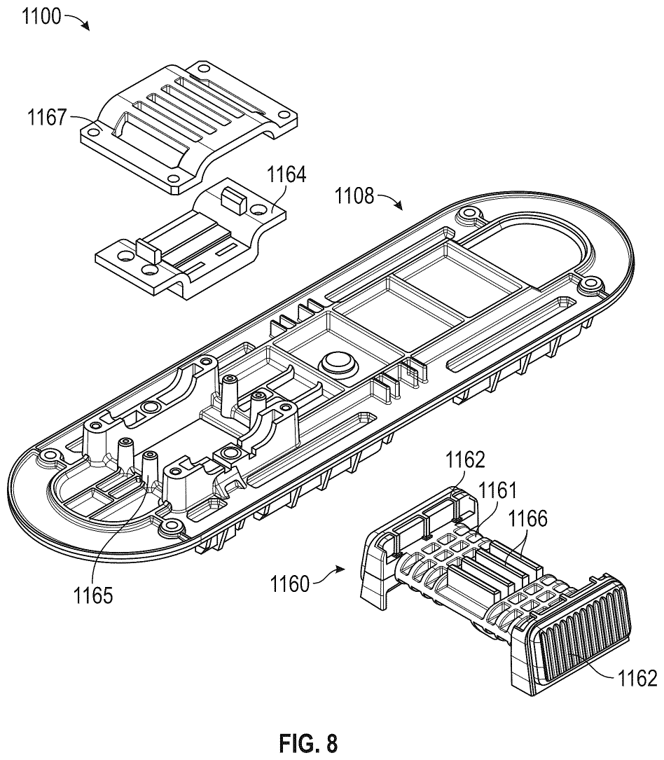

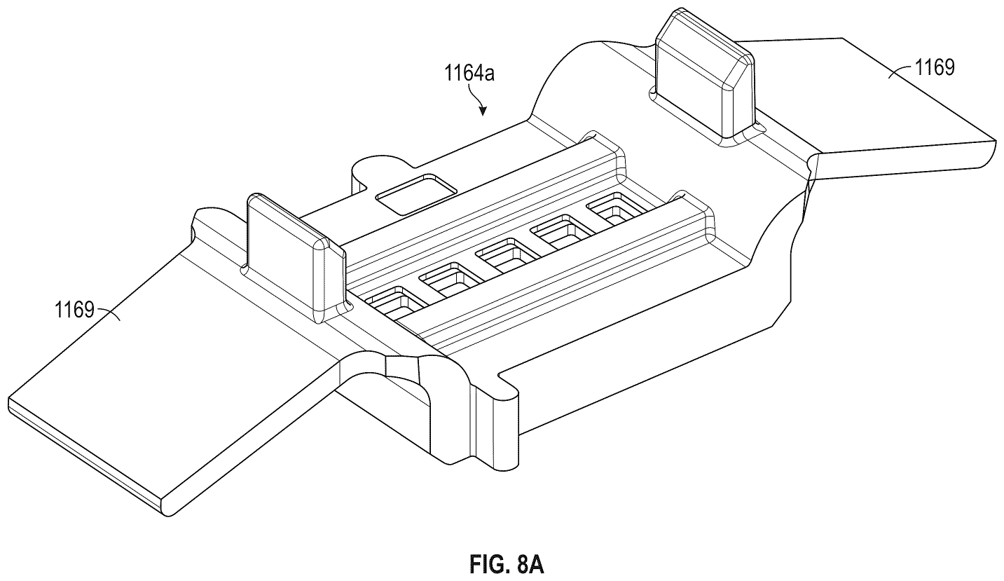

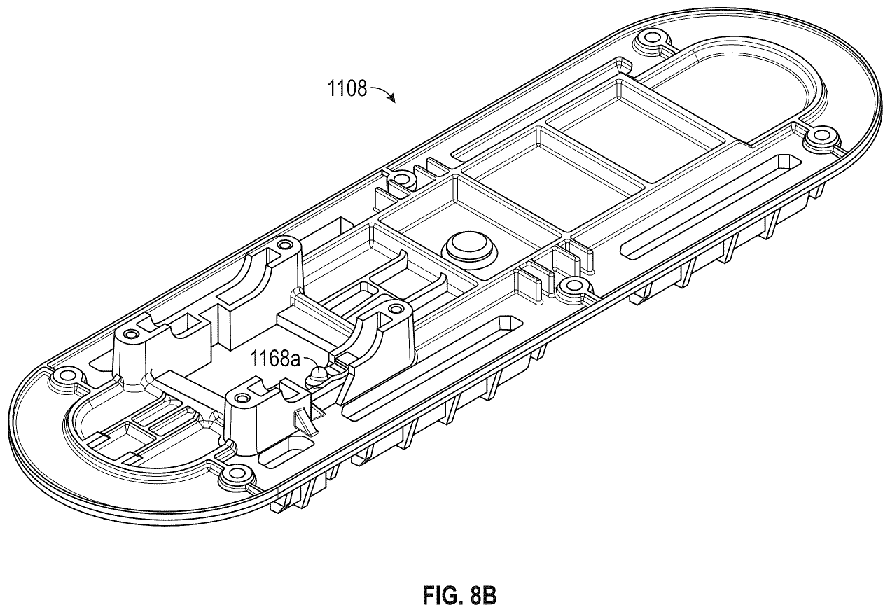

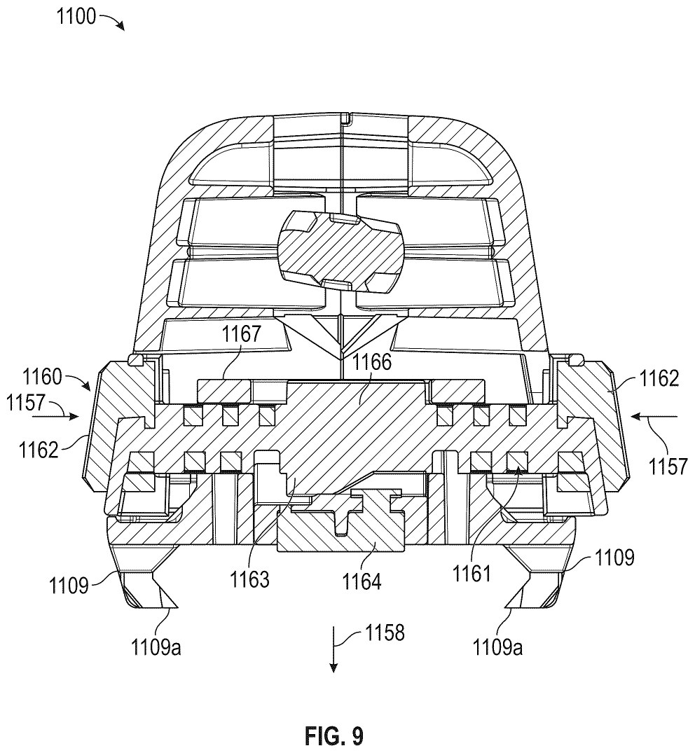

The rail dock 1100 can further include the brake system 1160. The brake system 1160 can include a toggle member 1161, as shown in FIGS. 7-10. The toggle 1161 can include two push buttons 1162. The push buttons 1162 can be on either end of the toggle 1161. The toggle 1161 can be actuated by pressing the appropriate button 1162 in a horizontal direction indicated by the horizontal arrows 1157 in FIG. 9. In some configurations, there is only one push button to toggle the brake system between a locked and unlocked configuration. Movement of the toggle 1161 in the horizontal direction can be guided by interaction of splines 1166 with a slotted member 1167. The toggle 1161 can be temporarily held in place by one or more spring loaded detent members 1168 in either position. In some embodiments, a detent member 1168a can be formed in one piece with the bottom plate 1108, e.g., by injection molding of plastic material. An example of the in-molded detent member 1168a is shown in FIG. 8B. The detent member 1168a extends from the surrounding structure of the bottom plate 1108 like a cantilever beam, and can flex under a force and recover its original position when the force is released. In some implementations, the button(s) 1162 may be substituted with alternative input or toggle members (e.g., slides, knobs, switches, and/or the like). In some configurations, the button(s) control gross movement of the rail dock 1100 along the rail 1002.

The toggle 1161 can include a ramp 1163. The ramp 1163 can be engaged or disengaged with a brake member 1164. The brake member 1164 can be actuated downwardly by the ramp 1163 and engage with the upper surface of the rail 1002, as indicated by the vertical arrow 1158 in Figure. 9. The brake member 1164 can comprise an clastic or other high-frictional material. The brake member 1164 engaged with the rail 1002 (such as on the upper surface thereof) can lock the position of the rail dock 1100 along the rail 1002 or at least inhibit translation along the rail during use (e.g., with a delivery system) due to friction between the brake member 1164 and the upper surface of the rail 1002. The brake member 1164 can be mounted on guide columns or posts 1165 that guide movement along a vertical direction (e.g., apertures in the brake member 1164 mounted over vertical guide columns or posts 1165, as shown in FIG. 8). The brake member 1164 can be biased out of engagement with the rail 1002 (e.g., by springs). For example, four springs, e.g., compression or leaf springs, can be disposed between the brake member 1164 and the top surface of the bottom plate 1108, one embracing each guide post 1165. In some embodiments, the brake member 1164 can be biased into the rail 1002.

Another embodiment of a brake member 1164a is shown in FIG. 8A, where the brake member 1164a comprises two flexible wings 1169 that are formed normally angled from a horizontal plane, e.g., parallel to the bottom surface of the brake member 1164a. In accordance with several embodiments, the installation of the brake member 1164a in the configuration illustrated in FIG. 8A does not need the guide posts 1165 shown in FIG. 8. When the brake member 1164a is pressed downwardly by the ramp 1163 to engage the upper surface of the bottom plate 1108 to stop the carriage assembly 1170, the flexible wings 1169 are biased to be flattened out, to positions more in parallel with the horizontal plane. The brake member 1164a may be made of a resilient material, e.g., injection molded plastic, so that once biased, the flexible wings 1169 have a tendency to recover their natural shape. As such, when the ramp 1163 is disengaged with the brake member 1164a, the brake member 1164a springs back up to the unbiased configuration and position. Since the position recovery of the brake member 1164a comes from the structure and the resilient material adopted, no spring may be needed. Thus, the brake member 1164a may be a springless brake. This may advantageously provide benefits of case of manufacturing and reduced cost.

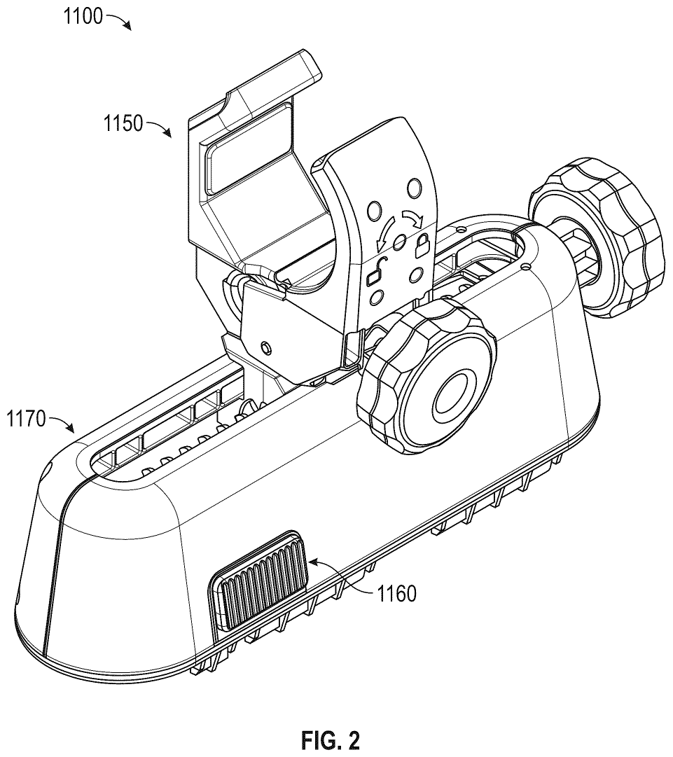

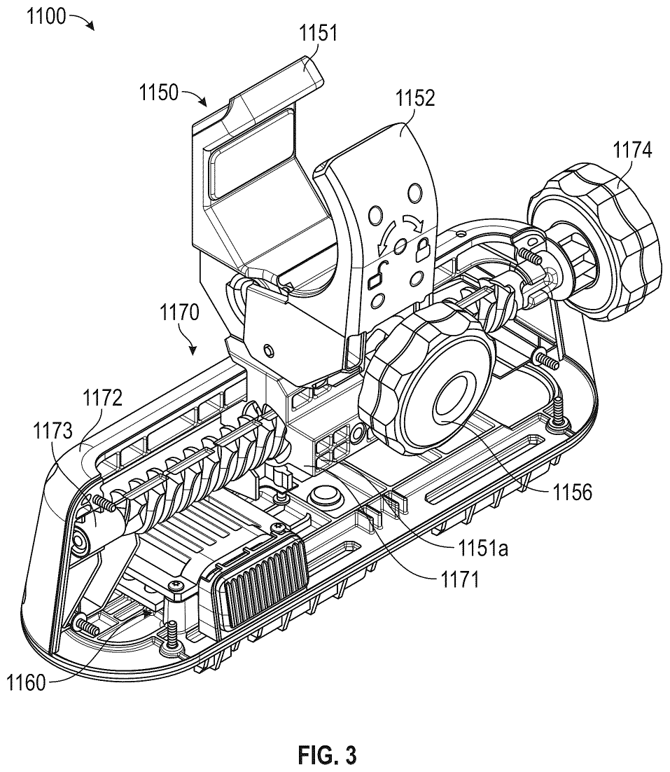

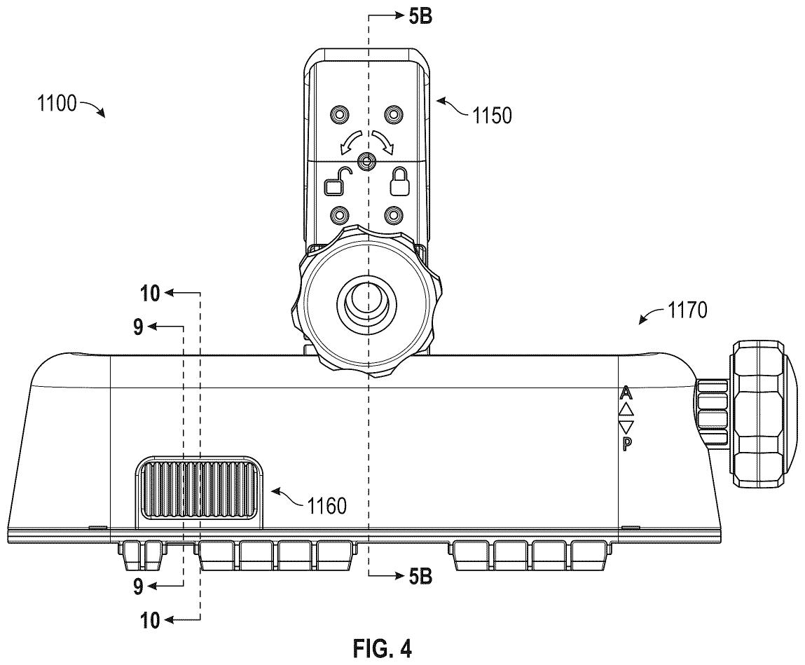

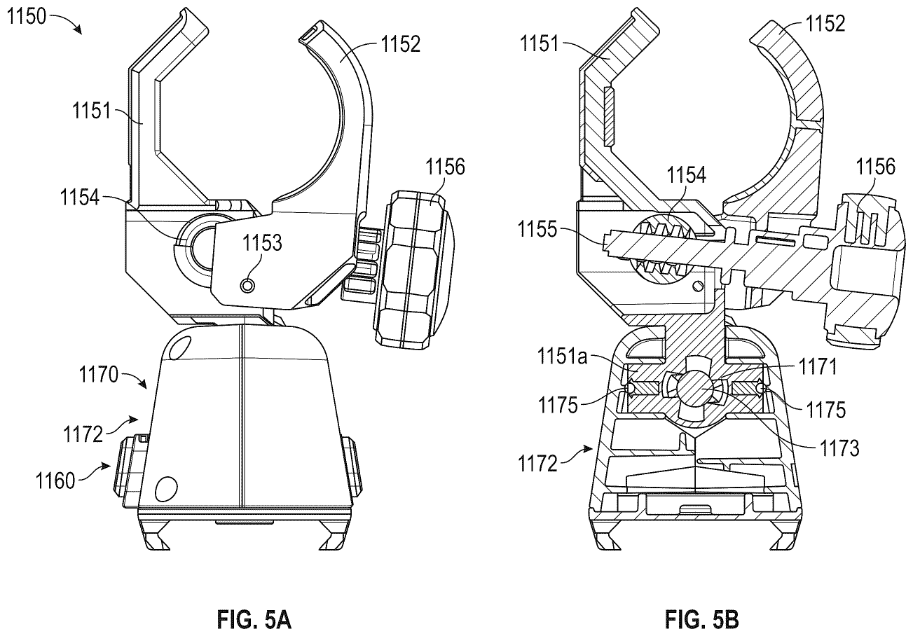

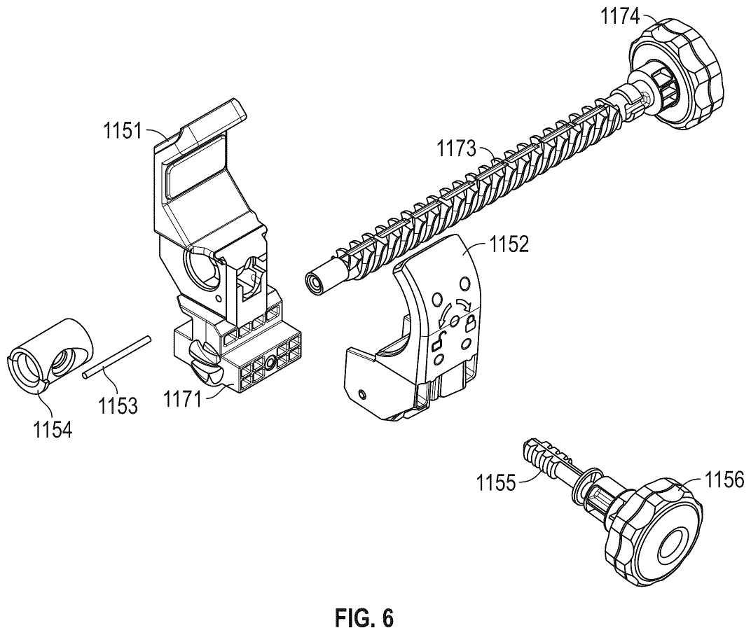

With reference back to FIGS. 2 and 4, the rail dock 1100 can include the support 1150 which can be used to attach a delivery system holder to the rail 1002. As shown, for example, in FIGS. 3, 5A-B, and 6, the support 1150 can include a fixed member 1151 and a movable member 1152. The movable member 1152 can be pivotably connected with the fixed member 1151 at a pin 1153. A lower end of the fixed member 1151 can be mounted on a carriage member 1171 of the carriage assembly 1170. The lower end of the fixed member 1151 include protrusions 1151a that fit within stabilizing slots of a housing 1172 of the carriage assembly 1170. An upper end of the fixed member can include a concave grip member opposite a corresponding concave grip member on an upper end of the movable member 1152. The concavity can be sized to receive a portion of a handle of the delivery system 2.

The support 1150 can include a nut 1154 and a shaft 1155 connected with a knob 1156. The nut 1154 can be disposed within the fixed member 1151. The shaft 1155 can be disposed within the movable member 1152. A threaded end of the shaft 1155 can be engaged with threads of the nut 1154 such that rotation of the knob 1156 can actuate the movable member 1152 between open and closed configurations. The threads on the shaft 1155 can provide for quickly actuating the movable member 1152 between the open and closed configurations. For example, the threads can facilitate opening/closing with rotation of the knob 1156 that is 180° (half turn) or less, or 90° (quarter turn) or less. In some implementations, the knob 1156 can be tightened past a locking point. Tightening the knob 1156 past the locking point can secure the members 1151, 1152 together in the closed configuration with greater force against inadvertent opening. In another example, the knob 1156 can be completely turned (360°) to arrive in the closed configuration.

The carriage assembly 1170 can include the housing 1172. The housing 1172 can enclose a travel screw or carriage shaft 1173. The carriage shaft 1173 can be mounted horizontally or at an angle and be rotatably about its axis by a knob 1174. The carriage 1171 (and the fixed member 1151) can be threadingly or threadedly mounted on the carriage shaft 1173 and translatable along the length of the carriage shaft by rotation of the knob 1174. Optionally, the carriage shaft 1173 can be lockable at intervals with one or a pair of opposing spring detent mechanism 1175 that can engage corresponding faces or concavities on the carriage shaft 1173. In some implementations, rotation of the knob 1174, and thus travel screw or carriage shaft 1173 controls fine movement of the rail dock 1100.

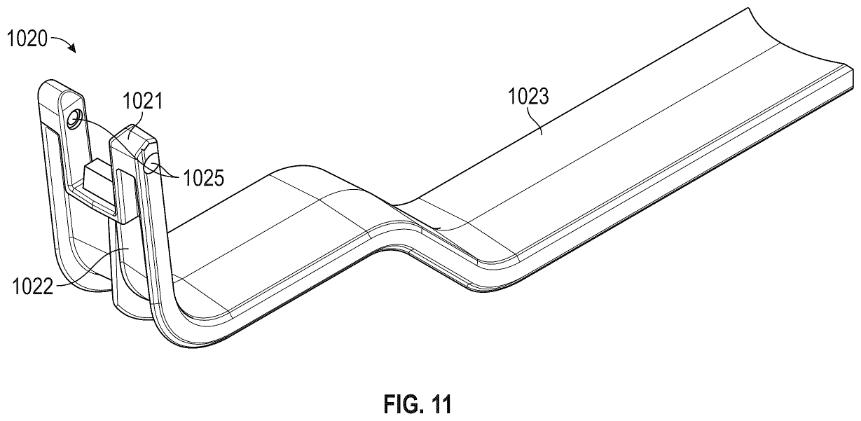

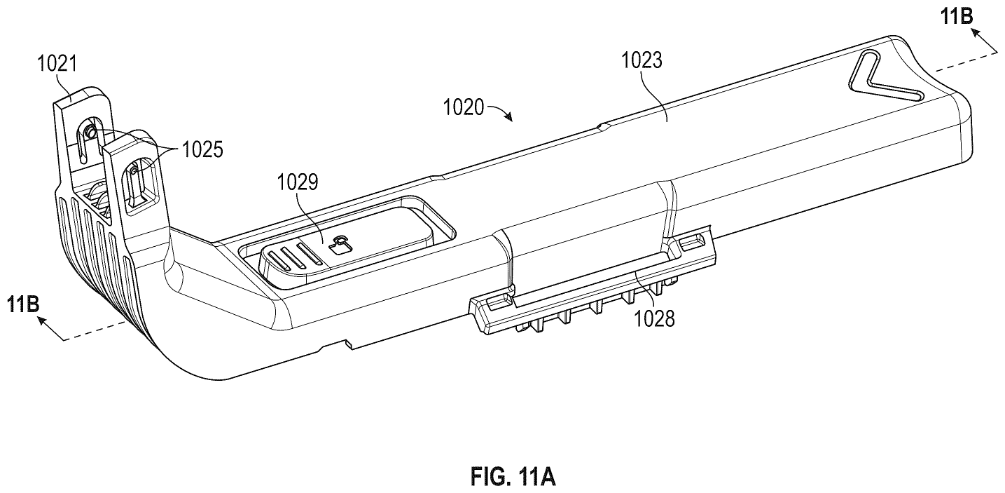

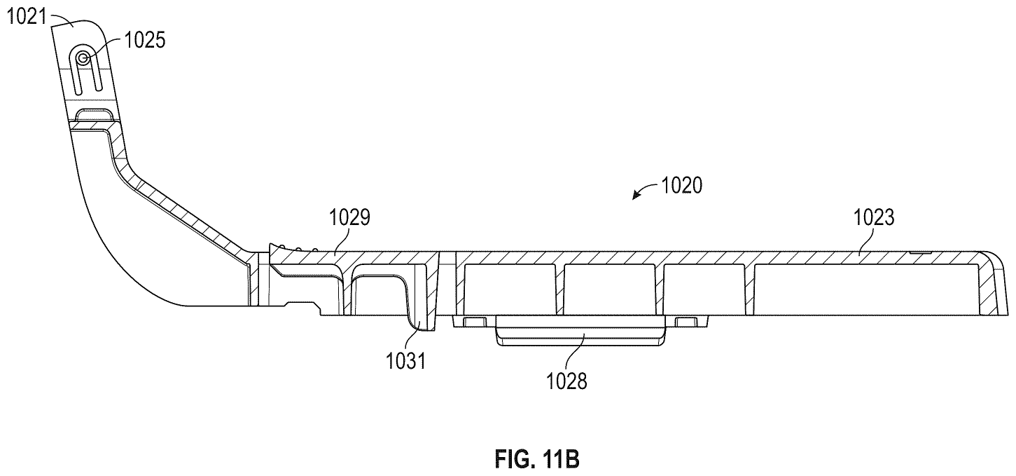

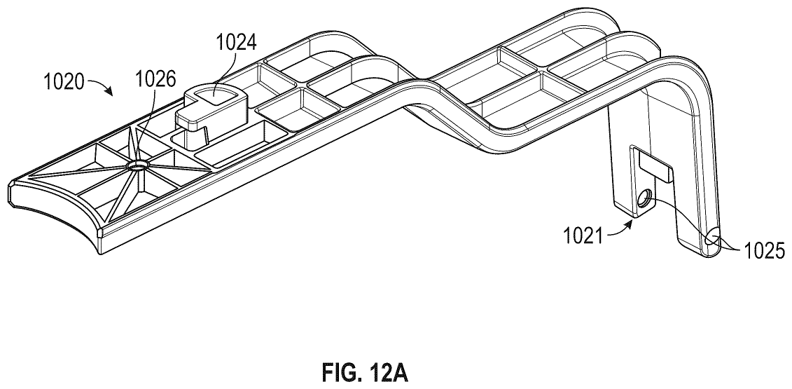

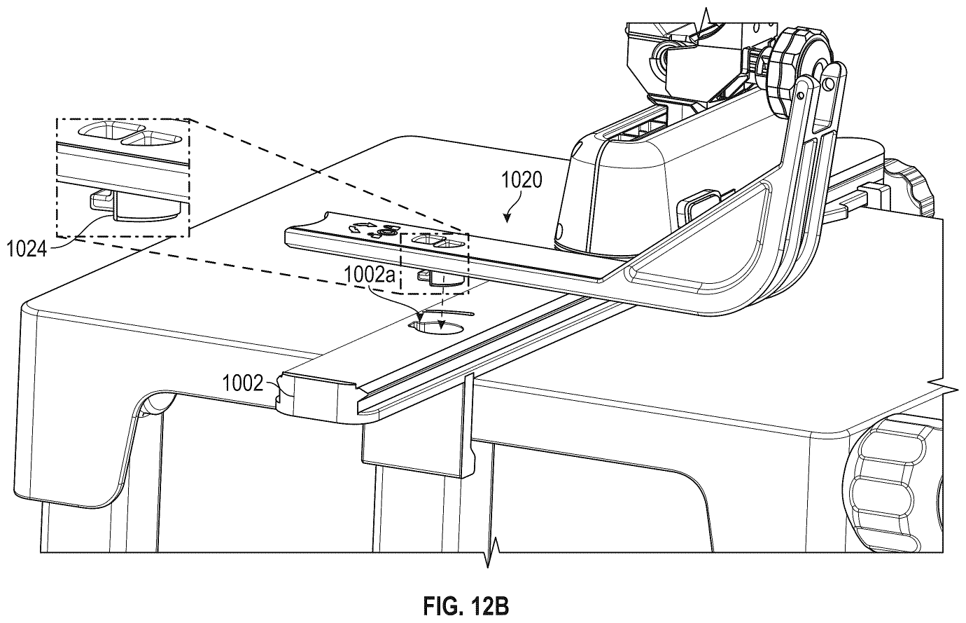

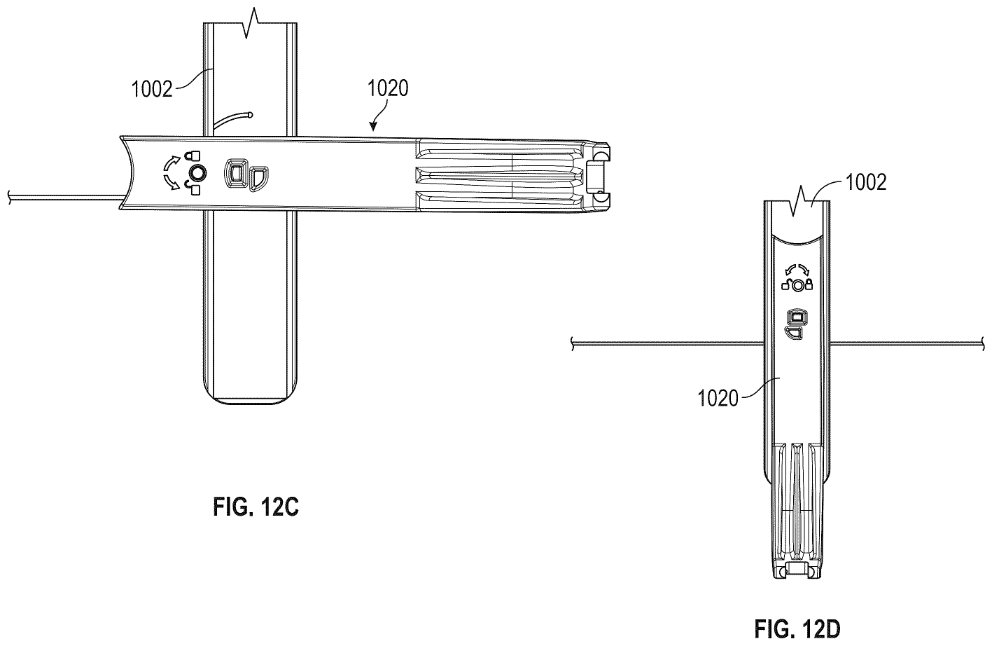

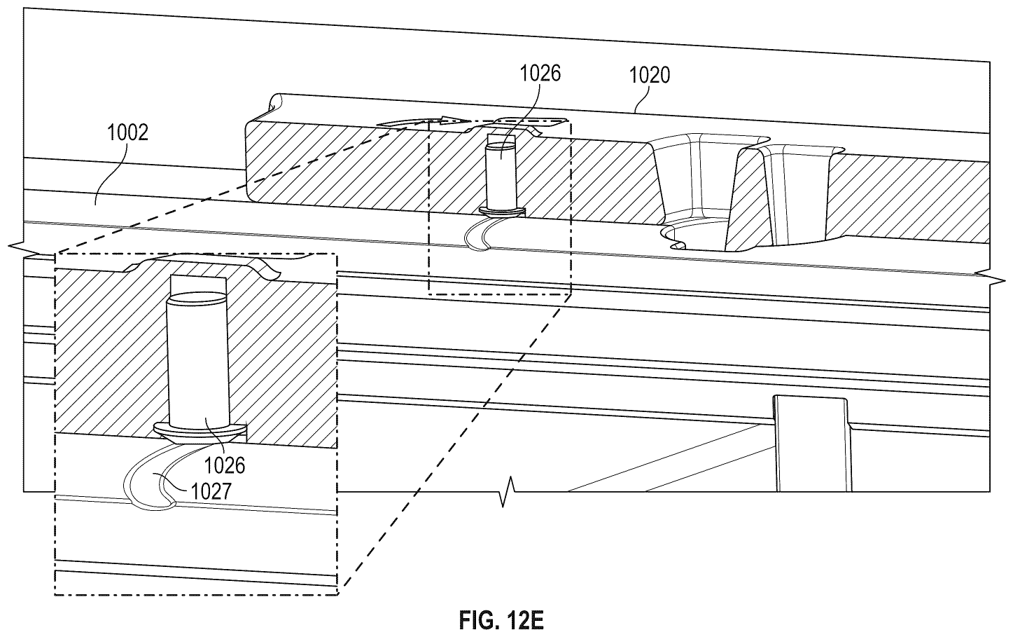

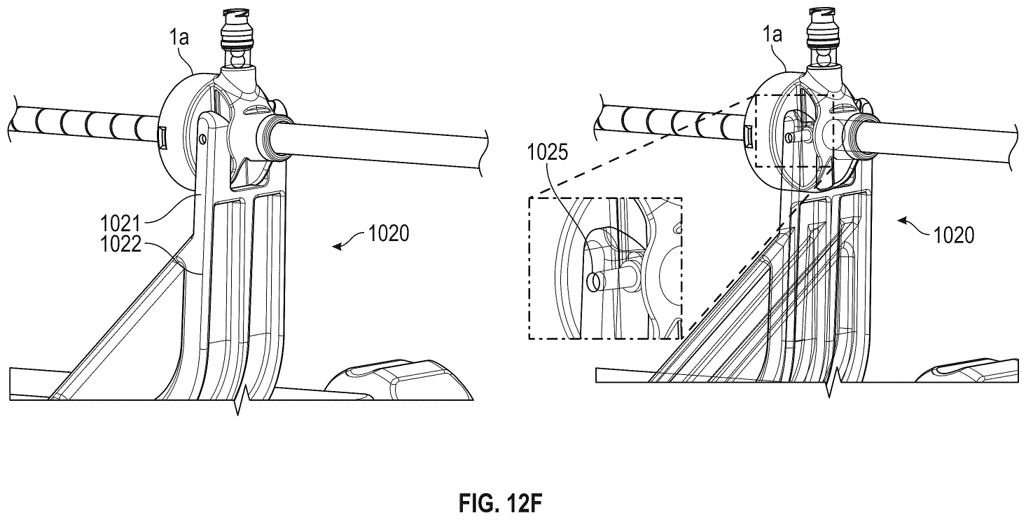

As shown in FIGS. 1A-1E, the stabilizer 1000 can include a hub support or hub nest 1020. The hub nest 1020 can be located distally to the rail dock 1100. As shown further in FIGS. 11-12F, the hub nest 1020 can include a support 1021 (such as a forked support) for engaging or supporting a portion of an introducer hub or sheath hub la, as shown in FIGS. 12F, or a portion of a handle or catheter of a delivery system 2, such as a shaft hub. The support 1021 can be located on a vertical portion 1022 of the hub nest 1020. The support 1021 can include one or more detents for securing the introducer hub or sheath hub la within the support 1021. A first detent can be located on a first fork of the support 1021 and a second detent can be located on a second fork of the support 1021. The detents 1025 can be spring-loaded. The detents 1025 can extend inwardly to engage with respective receiving portions (e.g., concavity) of the introducer hub or sheath hub la. In some embodiments, the detents 1025 may be formed as part of the hub nest 1020, e.g., injection molded in one piece when the hub nest 1020 with the detents 1025 is made of plastic, as shown in FIGS. 11A and 11B.

A horizontal portion 1023 of the hub nest 1020 can attach to an upward facing surface of the rail 1002. The horizontal portion 1023 can include a locking protrusion 1024 (FIG. 12A). The locking protrusion 1024 can include a transverse flange portion spaced from the horizontal portion 1023. The locking protrusion 1024 can be shaped to lock within a corresponding locking aperture 1002a in the rail 1002 (FIG. 12B). The locking aperture 1002a can be keyhole-shaped to accommodate the transverse flange of the protrusion 1024. The hub nest 1020 can be locked in place by insertion of the locking protrusion 1024 within the aperture 1002a (FIG. 12C) and rotation of the horizontal portion 1023 into alignment along the rail 1002 to engage the transverse flange with the rail 1002 (FIG. 12D). The hub nest 1020 can be removed by rotation in the opposite direction and withdrawing the locking protrusion 1024 from the aperture 1002a. Optionally, the rail 1002 can include multiple apertures 1002a for selectively mounting the hub nest 1020. The horizontal portion 1023 can include a detent 1026 (FIG. 12A). The detent 1026 can engage within a groove 1027 or aperture within the rail 1002 to secure the hub nest 1020 in the locked configuration (FIG. 12E). The detent 1026 can be spring-loaded.

Another embodiment of the hub nest 1020 is shown in FIGS. 11A and 11B, which is the hub nest 1020 assembled in stabilizer 1000 in FIGS. 1A, 1C, and 1D. The hub nest 1020 in FIGS. 11A and 11B comprises a first side embracing member 1028 extending from one side of the horizontal portion 1023 of the hub nest 1020 and a second embracing member 1028 extending from the other side of the horizontal portion 1023. Each of the embracing members 1028 has a distal end that overhangs the lower facing surface of the rail 1002. As such, removal of the hub nest 1020 from the rail 1002 in the vertical direction is prevented. The hub nest 1020 can be slid on to the rail 1002, e.g., from the distal end in the longitudinal direction of the rail 1002, allowing the embracing members 1028 to engage with and wrap around the side edges of the rail 1002. In this way, there is no need to rotate (e.g., quarter turn) when assembling the hub nest 1020 onto the rail 1002, which may result in simpler operation and reduced manufacturing cost, in accordance with several implementations.

The hub nest 1020 further comprises a locking mechanism 1029 (e.g., a slide-to-lock mechanism). When the locking mechanism 1029 is pressed downward, the protrusion 1031 can be levered up. This leverage effect is provided because the locking mechanism 1029 is pivotally coupled to the surrounding structure of the horizontal portion 1023. The leverage action allows the hub nest 1020 to further slide longitudinally toward the proximal end of the rail 1002, until the protrusion 1031 drops into the locking aperture 1002a in the rail 1002. As such, the hub nest 1020 is locked in place.

The locking mechanism 1029 may be injection molded in one piece with the hub nest 1020 when they are made of resilient plastic material. The coupling of the locking mechanism to the hub nest 1020 may result from thin connections between the locking mechanism 1029 and the surrounding structure of the horizontal portion 1023 formed about the locking mechanism 1029. The aperture 1002a may have a shape formed to fit the protrusion 1031. There may be more than one locking aperture 1002a in the rail 1002 to position the hub nest 1020 in different locations.

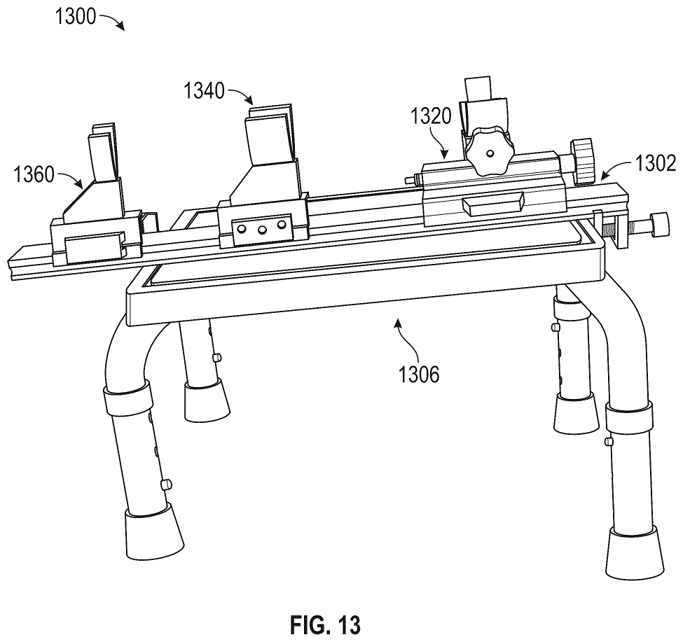

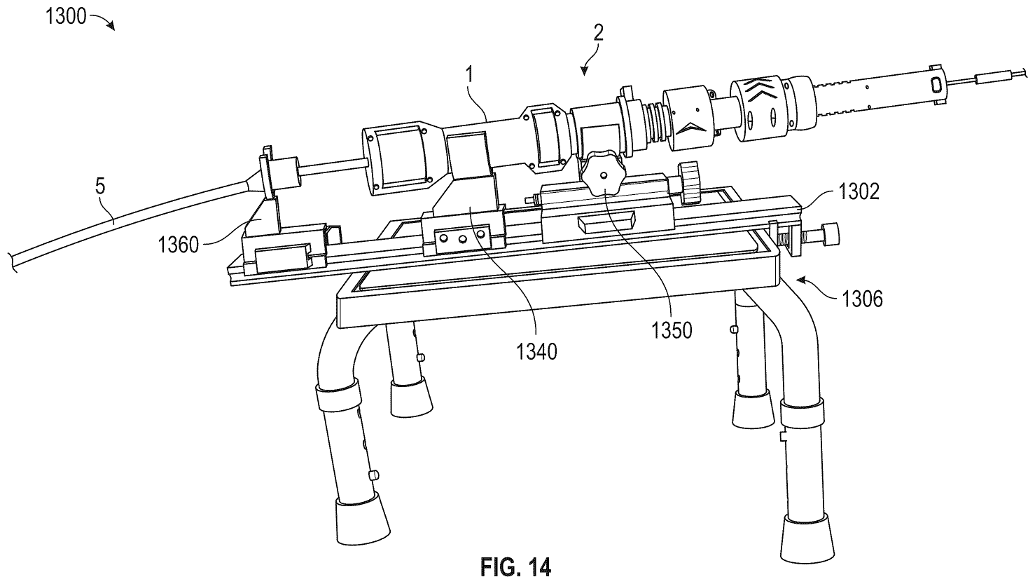

FIGS. 13-14 show another embodiment of a stabilizer 1300 for supporting a handle 1 of a delivery system 2. The stabilizer 1300 can be like stabilizer 1000 with the differences noted below. The stabilizer 1300 can include a base 1306 to which attaches a rail 1302 (e.g., by one or more clamps). The stabilizer 1300 can include one or more supports 1320, 1340, 1360 for the handle 1. The number and type of supports can vary based on the dimensions and functions of the handle 1.

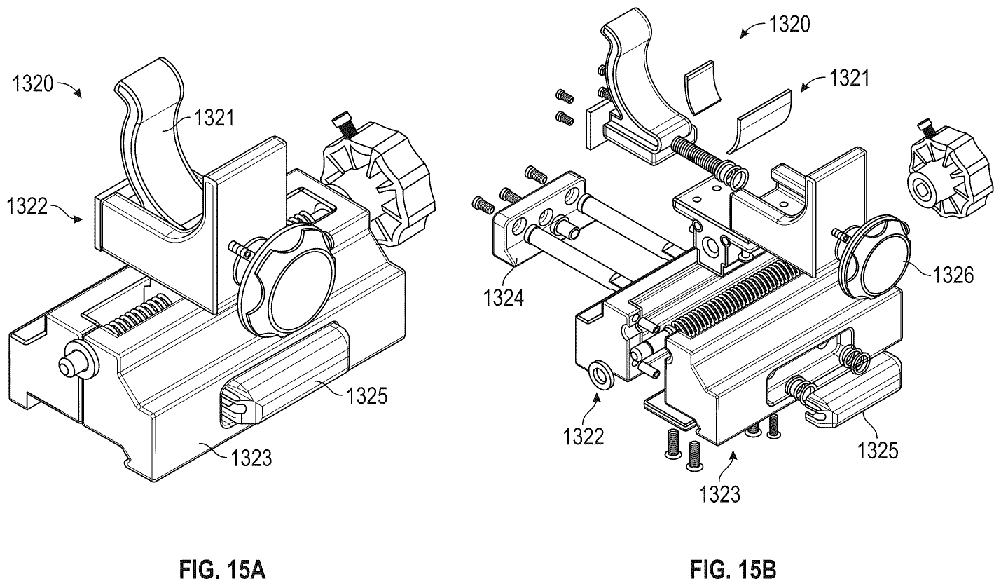

As shown in FIGS. 15A-B, the support 1320 can include a clamp 1321, a carriage 1322, and/or a rail dock 1323. The clamp 1321 can include a moveable member and fixed member and be actuated with a screw rod and knob 1326. By turning the knob 1326, the clamp can be opened or tightened to release or grip on the handle 1. The rail dock 1323 can include projections on a first side and a second side for engaging with a rail. The projection on the second side can be on a moveable plate 1324. The plate 1324 can be biased into the rail. The rail dock 1323 can be actuated between locked and unlocked configurations with a button 1325 coupled with the moveable plate 1324. In the locked configuration, the rail dock 1323 can be fixed along the rail. In the unlocked configuration, the rail dock 1323 can be moveable along the rail 1302 and/or removable from the rail 1302 in a vertical direction.

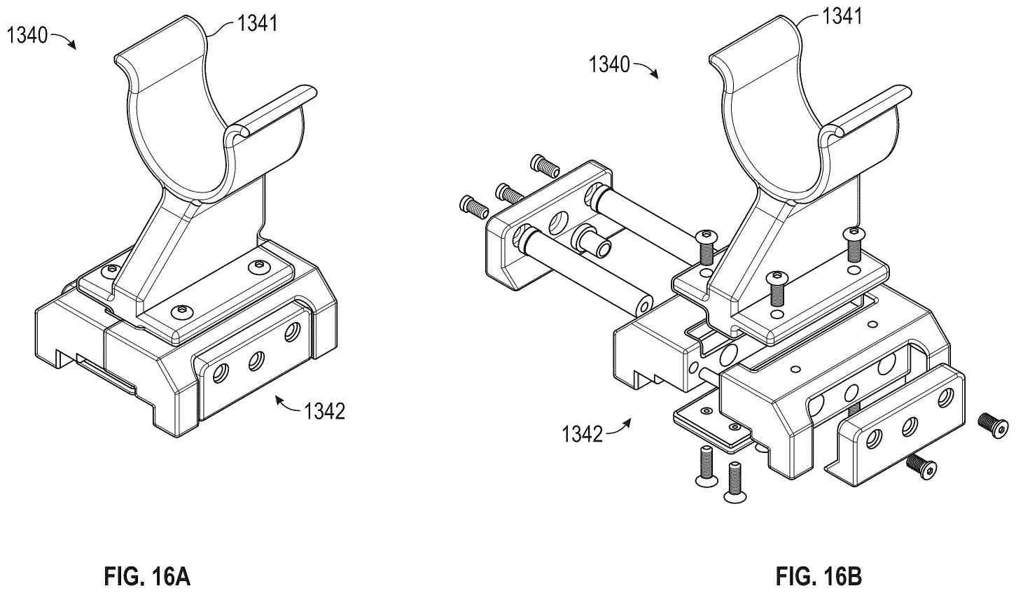

As shown in FIGS. 16A-B, the support 1340 can include a passive support 1341. The passive support 1341 can include two upward extending flexible members that flex outwardly to receive the handle 1 therebetween in a semi-secure manner. The support 1340 can further include a rail dock 1342, like the rail dock 1323. Optionally, the rail dock 1342 can be fixed in place by mechanical attachment (e.g., screws) of two opposing plates each having rail engagement projections.

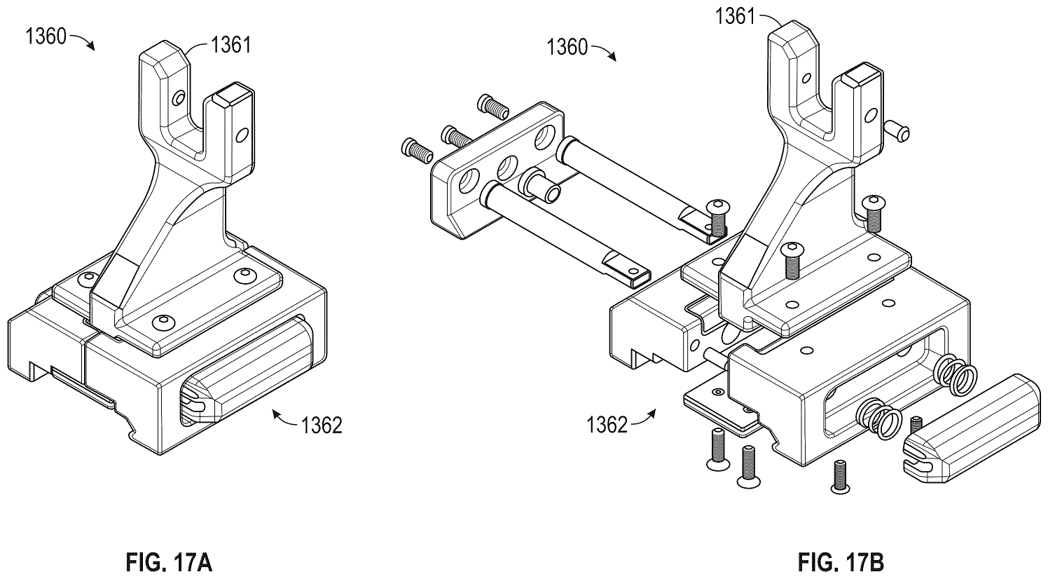

As shown in FIGS. 17A-B, the support 1360 can include a hub nest 1361 and a rail dock 1362, like the rail dock 1323.

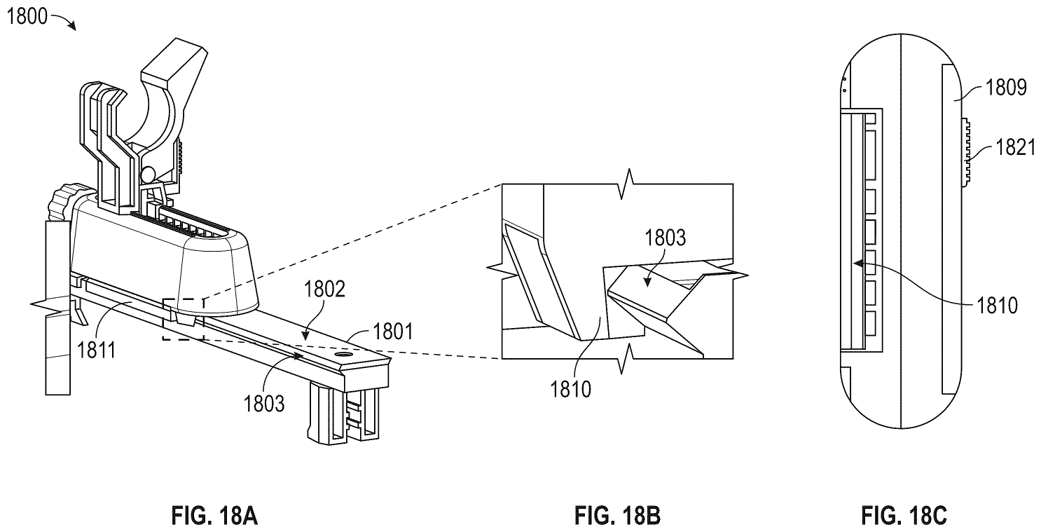

FIGS. 18A-C illustrate another rail dock 1800. Like the rail dock 1100, the rail dock 1800 can include a support or clamp for a delivery system, a carriage, and/or brake assembly 1811 that attaches with a rail 1802. The brake assembly can include a first side with a first projection 1809 that can overhang a first side 1801 of the rail 1802. A second side of the brake assembly can include a second projection 1810. The second projection 1810 can overhang a second side 1803 of the rail 1802 opposite the first side. The second projection 1810 can be biased (e.g., via spring) into the second side 1803 of the rail 1802 by a force and actuatable between locked and unlocked configurations (e.g., via a button 1821). The second projection 1810 can inhibit removal of the rail dock 1800 from the rail 1802 in the vertical direction, but still allow translation along the rail in the unlocked configuration. The first and/or second sides of the rail 1802 can be textured (e.g., knurled) to increase friction with the projections 1809, 1810 and enhance braking power.

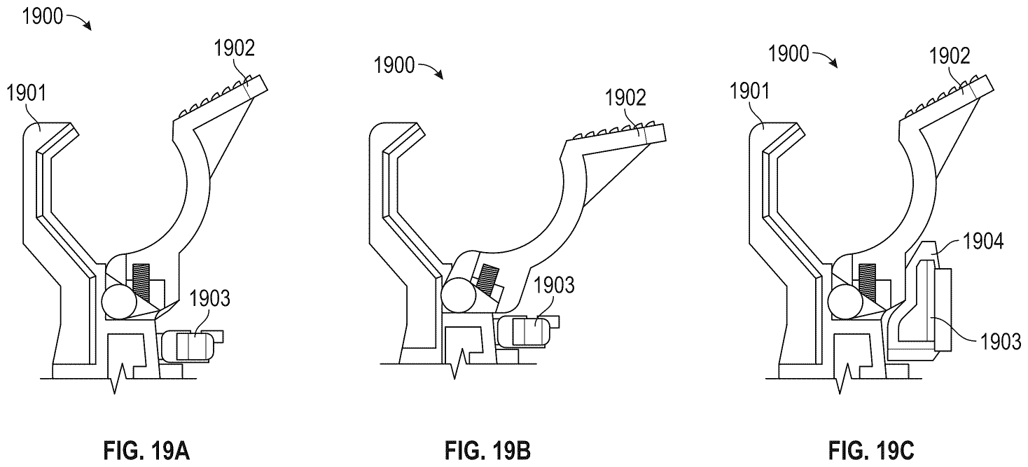

FIGS. 19A-C illustrate a support 1900. The support 1900 can be used as a support for a delivery system and used in conjunction with a rail dock, e.g., one of the rail docks discussed above. The support 1900 can include a fixed member 1901 connected with a movable member 1902. Upper ends of the fixed and movable members 1901, 1902 can be concave to receive the delivery system. The support 1900 can be actuatable between an open configuration (FIG. 19B) and a closed configuration (FIGS. 19A, 19C) based on a position of the movable member 1902. A handle (e.g. handle 1) of the delivery system (e.g., delivery system 2) can be clamped in the space defined by the fixed member 1901 and the movable member 1902 when the support 1900 is locked at the closed configuration. The handle can be loaded into or removed from the space between the fixed member 1901 and the movable member 1902 when the support 1900 is at the open configuration.

The support 1900 can include a rotatable lock 1903 that is rotatable about an axis. The axis can be transverse to a pivot axis of the movable member 1902. The rotatable lock 1903 can include an extension portion 1904. The rotatable lock 1903 can be actuatable between an unlocked position (FIGS. 19A-B) that permits opening of the movable member 1902 and a locked position (FIG. 19C) that blocks opening of the movable member 1902. The extension portion 1904 in the locked position can secure the movable member 1902 in the closed configuration.

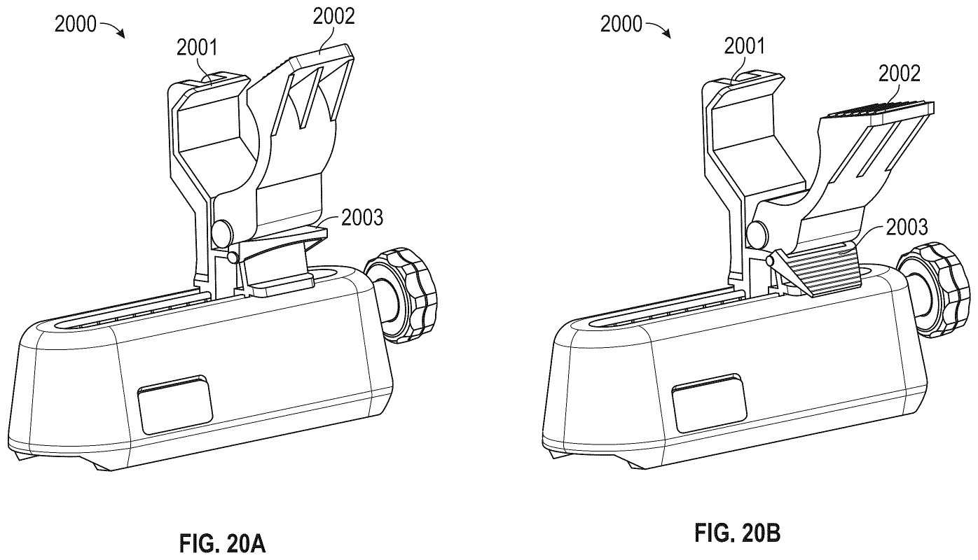

FIGS. 20A-B illustrate a support 2000. The support 2000 can be used as a support for a delivery system and used in conjunction with a rail dock. The support 2000 can include a fixed member 2001 connected with a movable member 2002. Upper ends of the fixed and movable members 2001, 2002 can be concave to receive the delivery system. The support 2000 can be actuatable between an open configuration (FIG. 20B) and a closed configuration (FIG. 20A) based on a position of the movable member 2002. The support 2000 can include a rotatable lock 2003 that is rotatable about an axis. The axis can be transverse or parallel to a pivot axis of the movable member 2002. The rotatable lock 2003 can be actuatable between an unlocked position (FIG. 20B) that permits opening of the movable member 2002 and a locked position (FIG. 20A) that blocks opening of the movable member 2002.

FIG. 21 shows a support 2100 for holding a handle 1 of a delivery system 2. The support 2100 can be mounted on a rail dock. The support 2100 can include a lower portion 2101 and a strap 2102. The handle 1 of the delivery system 2 can be supported on the lower portion 2101, which may include a concave region. The strap 2102 can be fixed on a first end (not shown) and a second end 2103 can wrap around the handle 1 and be secured with the lower portion 2101. The strap 2102 can comprise an elastic or compliant material. The second end 2103 can include an aperture that is received over a protrusion 2104 on the lower portion 2101. The second end 2103 may include multiple apertures or positions to adjust a tension on the strap 2102. For example, one position can prevent removal of the handle 1 and allow rotation, and second position can prevent rotation. Alternatively, the position of the protrusion 2104 can be adjustable. For example, by adjusting the position of the protrusion 2104, the tension on the strap 2102 can be selected or adjusted to allow rotation of the handle 1 about a longitudinal axis within the support 2100. Alternatively, the second end 2103 can be T-shaped and locked under a shelf or overhang on the support 2100.

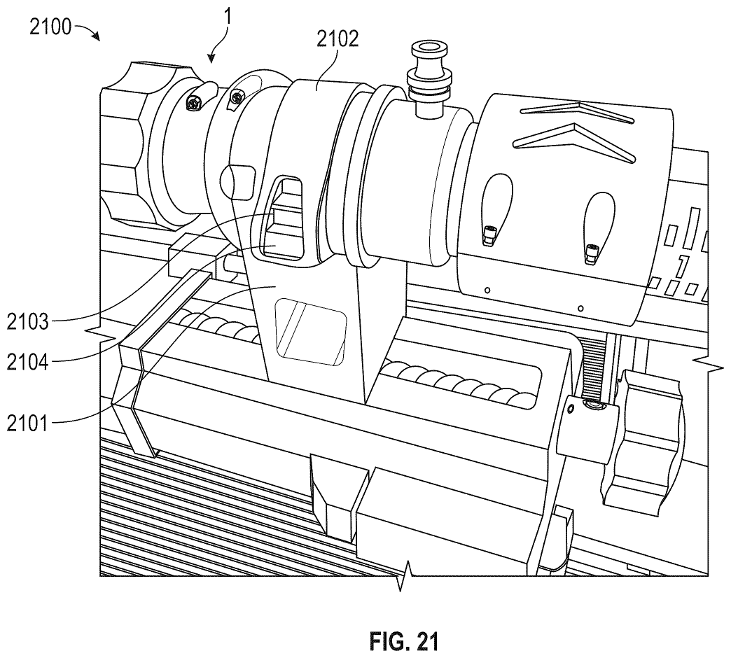

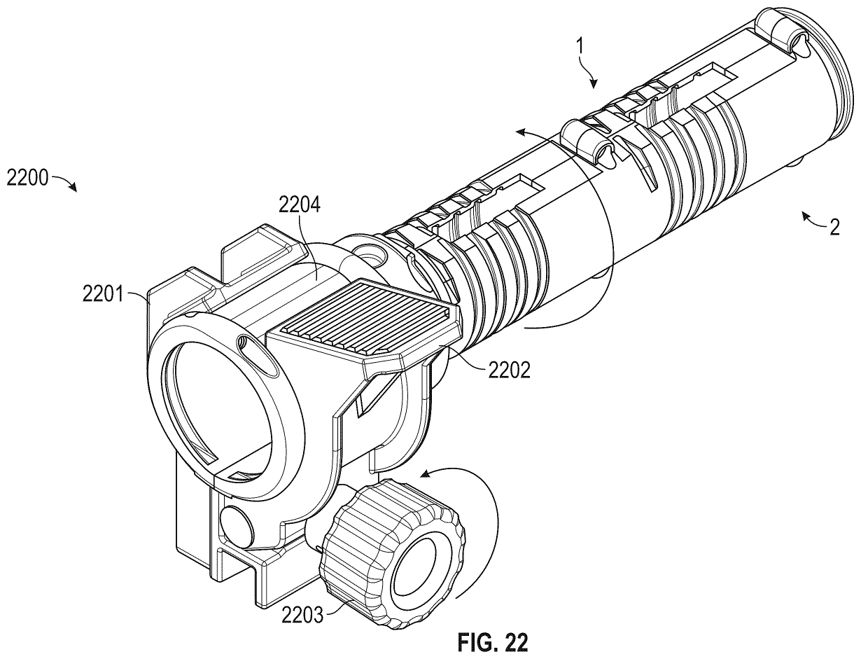

FIG. 22 shows a support 2200 for a handle 1 of a delivery system 2. The support 2200 can be mounted on a rail dock. The support 2200 can include a clamp that engages at least partially about a circumference 2204 of the handle 1. The clamp can have a fixed member 2201 and a movable member 2202. The clamp can be locked in place about the handle 1. The support 2200 can further include a knob 2203 connected with a worm gear (not shown). The worm gear can engage with threads that extend about the circumference 2204 of the handle 1. Accordingly, rotation of the knob 2203 can cause rotation of the handle 1 about a longitudinal axis while being held in the clamp. The threads of the handle 1 can be integral with a housing thereof or, alternatively, the clamp can include a separate worm wheel member that can be attached about the handle 1. The support 2200 or handle 1 can include positional indicators to show the angle of adjustment.

The various embodiments of the supports of the various stabilizers disclosed above are configured to hold the delivery system 2 stably during a medical procedure. For example, in FIG. 1A, the handle 1 of the delivery system 2 is held in support 1150, and the introducer hub or sheath hub 1a attached to the guidewire of the delivery system 2 is held by the hub nest 1020 (another support). As shown in FIG. 14, the handle 1 of the delivery system 2 is held by two supports 1350 and 1340. The introducer hub or sheath hub 1a is held by support 1360. The location of each support on the rail 1002, 1302 of the stabilizer 1000 can be adjusted to fit the handle and then locked in place. The gripping tightness can be adjusted to ensure that the handle 1 and the delivery system 2 are stably held in place. The introducer hub or sheath hub 1a may be a shaft hub through which one or more shafts of the delivery system 2 are inserted. The introducer hub may be coupled to an introducer sheath that is inserted within vasculature of a patient to facilitate introduction of the delivery system into the vasculature.

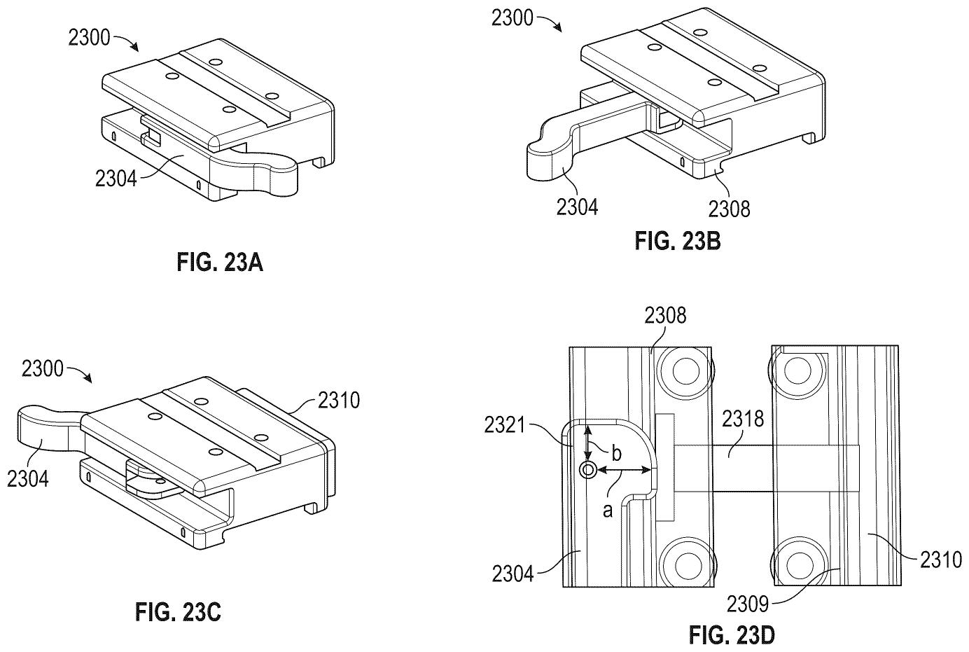

FIGS. 23A-D illustrate a rail dock 2300 that can be used with a support, e.g., one of the supports disclosed above, for a delivery system. The rail dock 2300 may have a bottom surface with a first side with a protrusion 2308 that at least partially wraps around a bottom surface of a rail for allowing the rail dock 2300 to slide along the rail, while preventing removal. The rail dock 2300 can include a movable plate 2310 on a second side of the bottom surface with a protrusion 2309. The movable plate 2310 can be connected with a lever lock 2304 for attachment and detachment with the rail and for maintaining the position of the rail dock 2300 on the rail.

As shown in the figures, the lever lock 2304 can include a handle rotatably connected to one side of the rail dock 2300 and attached, such as by a spring or pair of springs and/or a shaft 2318 or other attachment member to the movable plate 2310 on an opposite side of the rail dock 2300. The handle can fit within a cutout on a side of the rail dock 2300 in some embodiments. The lever lock 2304 can be rotated between a locked configuration (FIG. 23A) in which the protrusions 2308, 2309 engage with the sides of the rail and lock the rail dock 2300 in place, a semi-locked configuration (FIG. 23B) in which the protrusions prevent removal of the rail dock 2300 from the rail in the vertical direction and permit sliding along the rail, and an unlocked configuration (FIG. 23C) in which the protrusions allow removal of the rail dock from the rail in the vertical direction. A base 2321 of the lever lock 2304 can include different widths a, b, c, that each position the movable plate 2310 (coupled through the shaft 2318) corresponding to one of the locked, semi-locked, and unlocked configurations, respectively.

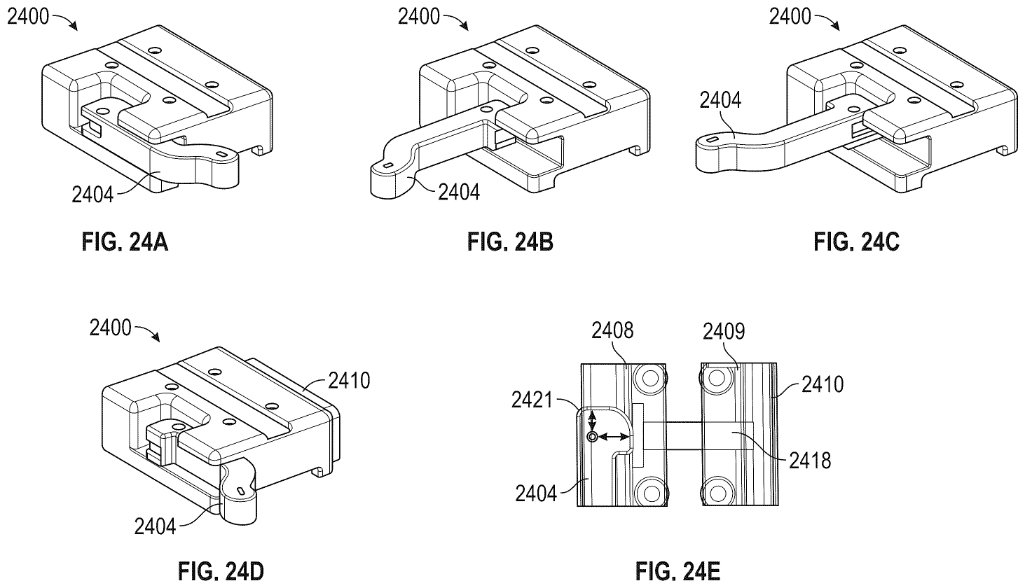

FIGS. 24A-E illustrate a rail dock 2400 that can be used with a support for a delivery system. The rail dock 2400 may have a bottom surface with a first side with a protrusion 2408 that at least partially wraps around a bottom surface of a rail for allowing the rail dock 2400 to slide along the rail, while preventing removal. The rail dock 2400 can include a movable plate 2410 on a second side of the bottom surface with a protrusion 2409. The movable plate 2410 can be connected with a lever lock 2404 for attachment and detachment with the rail and for maintaining the position of the rail dock 2400 on the rail.

As shown in the figures, the lever lock 2404 can include a handle rotatably connected to one side of the rail dock 2400 and attached, such as by a spring or pair of springs and/or a shaft 2418 or other attachment member to the movable plate 2410 on an opposite side of the rail dock 2400. The handle can fit within a cutout on a side of the rail dock 2400 in some embodiments. The lever lock 2404 can be rotated and flipped between a locked configuration (FIG. 24A) in which the protrusions 2408, 2409 engage with the sides of the rail and lock the rail dock 2400 in place, a semi-locked configuration (FIGS. 24B-C) in which the protrusions prevent removal of the rail dock from the rail in the vertical direction and permit sliding along the rail, and an unlocked configuration (FIG. 24D) in which the protrusions allow removal of the rail dock from the rail in the vertical direction. A base 2421 of the lever lock 2404 can include different widths a, b, c, that each position the movable plate 2410 (coupled through the shaft 2418) corresponding to one of the locked, semi-locked, and unlocked configurations, respectively. The rail dock 2400 shown in FIGS. 24A-E is similar to the rail dock 2300 shown in FIGS. 23A-D. The difference is that the rail dock 2400 has a cutout to expose the base 2421 of the lever lock 2404 but the rail dock 2300 does not.

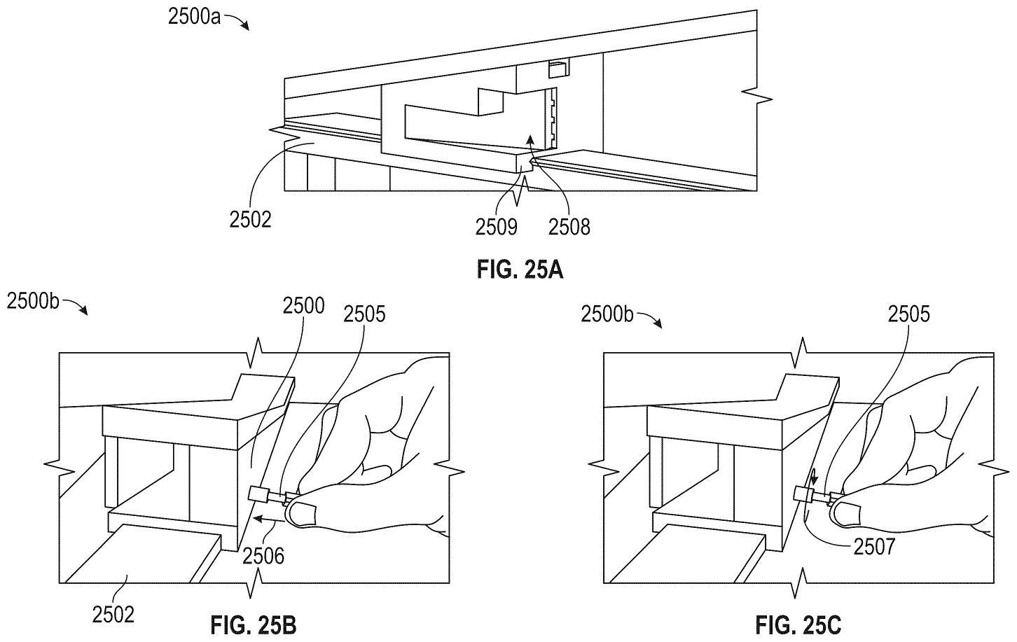

FIG. 25A illustrates another rail dock 2500a for mounting on a rail 2502. The rail dock 2500 can include a lever 2508 that overhangs one side of the rail. The lever 2508 can swing out and in to actuate a protrusion (e.g., button or nub 2509) between locked and unlocked configurations that prevent and permit movement of the rail dock 2500 along the rail 2502, like the disclosed rail docks of the stabilizers.

FIG. 25B-C illustrates another rail dock 2500b for mounting on a rail 2502. The rail dock 2500b can include a separate locking actuator 2505 to prevent removal of the rail dock 2500 from the rail 2502 in the vertical direction. The locking actuator 2505 can be a pin that is insertable within a side of the rail dock 2500 in the direction of arrow 2506 or rotated in the direction of arrow 2507. When inserted and/or rotated, the locking actuator 2505 can directly overhang one side of the rail 2502 (e.g., opposite the protrusion 2509) and thereby prevent removal in the vertical direction. Alternatively, the locking actuator 2505 can actuate a mechanism that directly overhangs the rail 2502.

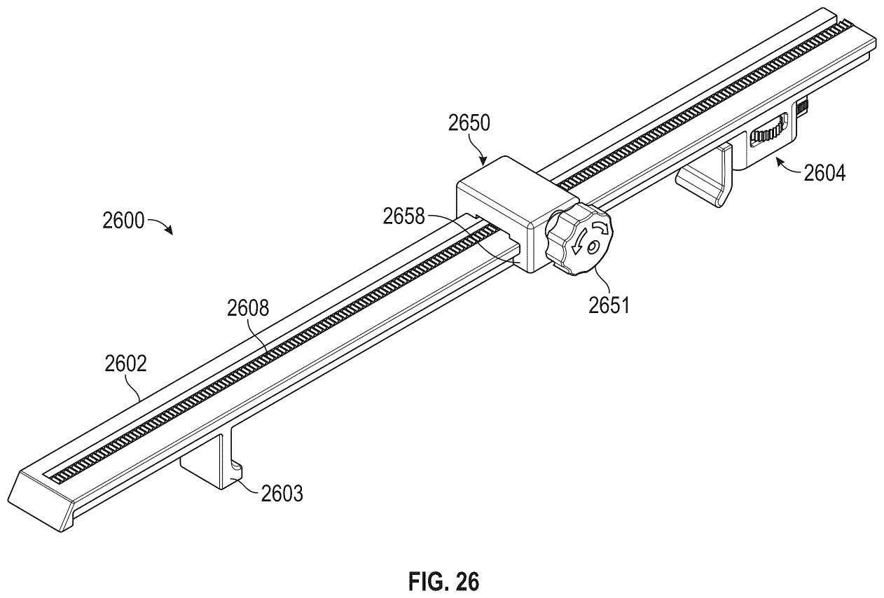

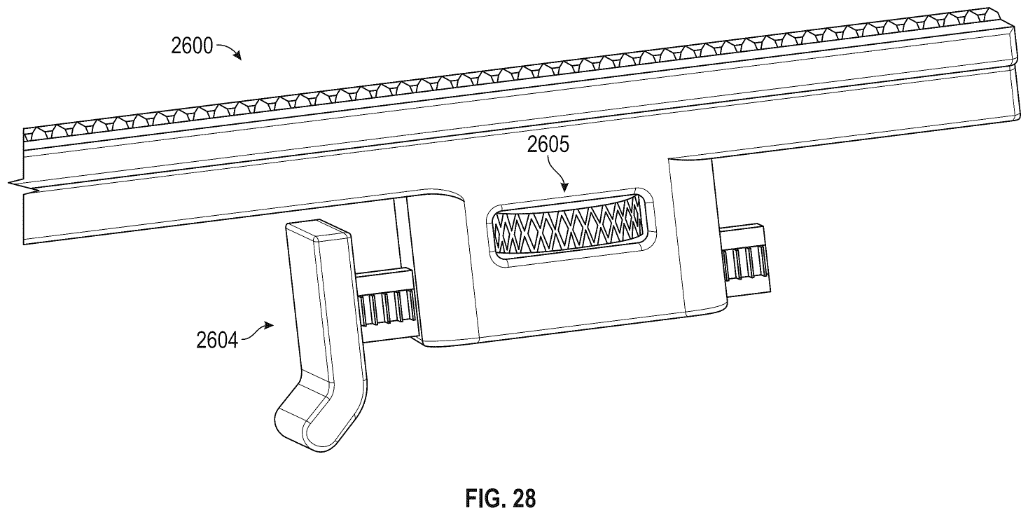

FIGS. 26-28 show another stabilizer assembly 2600. The stabilizer assembly 2600 can include a rail 2602. The rail 2602 can include a fixed clamp 2603 and a movable clamp 2604. One or both of the clamps 2603, 2604 can be integrated with the rail 2602. The rail 2602 can be attachable with a base by the clamps 2603, 2604. The movable clamp 2604 can include an adjustment wheel 2605 that actuates the movable clamp 2604 along a longitudinal direction of the rail 2602. The adjustment wheel 2605 can include a gear that engages with a rack along a side of the movable clamp 2604.

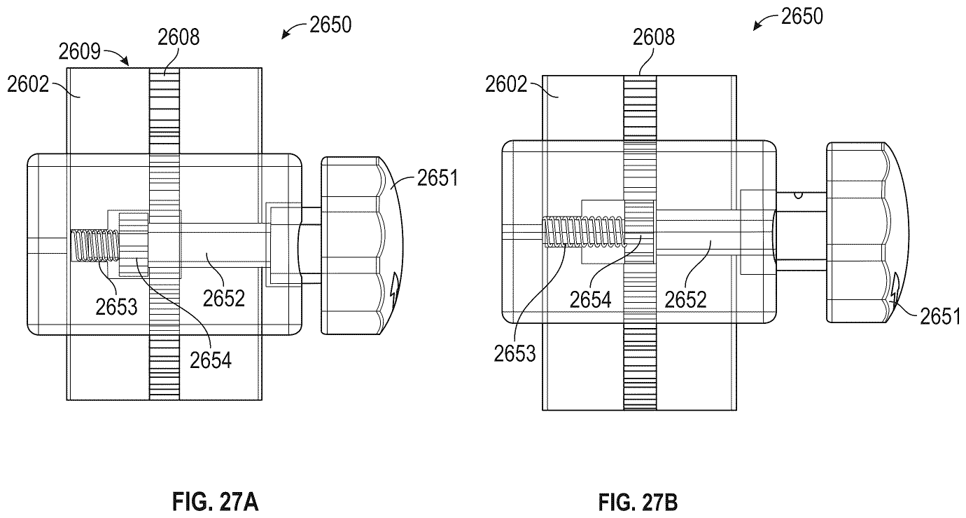

A rack 2608 comprising gear teeth can extend along a longitudinal direction between first and second ends of the rail 2602. The rack 2608 can be centered and/or on an upper surface of the rail 2602. The stabilizer 2600 can include a rail dock 2650. The rail dock 2650 can provide a base for a support for a delivery system. The rail dock 2650 can include opposing projections 2658 that overhang opposite sides of the rail 2602. The rail dock 2650 can be mountable on the rail 2602 at the first or second ends thereof. The rail dock 2650 can include a knob 2651 attached with a shaft 2652 and biased by a spring 2653. A pinion gear 2654 can be mounted on the shaft 2652. The pinion gear 2654 can be movable into and out of engagement with the rack 2608 based on a position of the shaft 2652 and knob 2651. As shown in FIG. 27A, when the knob 2651 is pushed towards the left side, the spring 2653 is compressed and the pinion gear 2654 is disengaged with the rack 2608. Then in FIG. 27B, the pushing force on the knob 2651 is released. The pinion gear 2654 is pushed rightward by the spring 2653 back to the position to engage the rack 2608.

When the pinion gear 2654 is engaged with the rack 2608, rotation of the knob 2651 can cause translation of the rail dock 2650 along the longitudinal direction relative to the rail 2602 (e.g., fine position adjustment). Torsional resistance in the rotation of the knob 2651 and/or shaft 2652 can prevent unintended back driving of the rail dock 2650. When the pinion gear 2654 is disengaged with the rack 2608 (and received within a well 2609 adjacent to the rack 2608), the rail dock 2650 can move freely along the longitudinal direction relative to the rail 2602 (e.g., gross position adjustment).

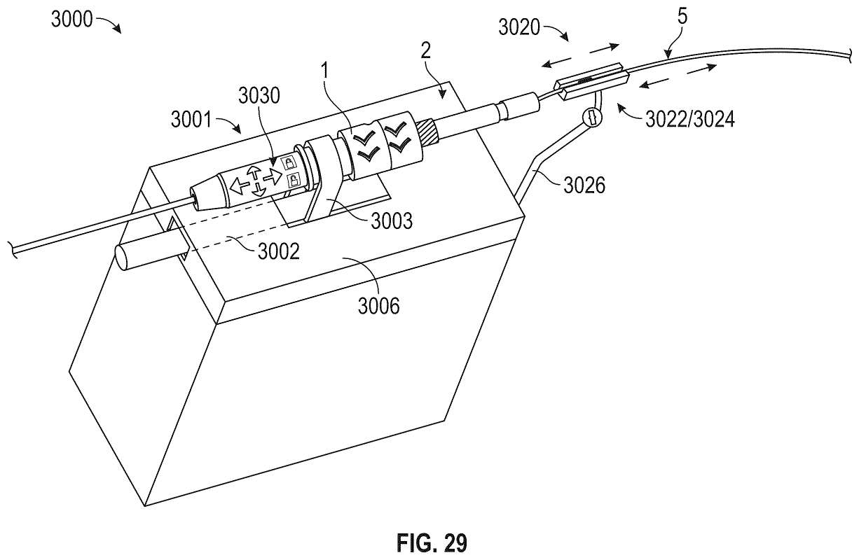

FIGS. 29-31 show a surgical catheter system 3000 including a handle 1 of a delivery system 2 mounted in a stabilizer assembly 3001. The stabilizer 3001 can include the features of any of the stabilizers disclosed herein. The stabilizer 3001 can include a support 3003 for the handle 1, a base 3006, and a rail 3002.

The delivery system 2 can include a shaft assembly with a proximal end and a distal end. As with the stabilizer 1000, the proximal end of the shaft assembly is toward the operator (e.g., clinician or healthcare professional), and the distal end is away from the operator. The proximal end of the shaft assembly attached with the handle 1. A distal portion of the shaft assembly can be inserted within a patient's body over the guidewire 5. A lumen can extend from the distal end of the shaft assembly to a proximal end of the handle. The guidewire 5 can be received within the lumen. A proximal section of the guidewire 5 can extend proximally from the handle 1. The guidewire can comprise a metal material or other suitable material. In one example, the guidewire 5 can include a solid core with a braided metal sheath.

In a conventional delivery system, one physician or other healthcare professional generally controls the delivery system 2 and a second physician or other healthcare professional controls the position of the guidewire 5 (e.g., by grasping the proximal portion of the guidewire 5). The position of the guidewire 5 may need to be manipulated based on the surgical steps required for an operation. For example, the guidewire 5 can be retracted to aid in crossover of a septum or turning a stiff section of the delivery system 2. The guidewire 5 can be advanced to add height to the position of the delivery system 2. All advancing, retracting or holding a static position of the guidewire 5 is done manually using current systems by the second physician or healthcare professional. Aspects of the present disclosure relate to an improved system that can function as a “third hand” to advantageously allow a single physician to position the guidewire 5 during an operation.

As shown in FIG. 31, the surgical catheter system 3000 can include a guidewire management system 3020. The guidewire management system 3020 can support, and optionally control and arrest movement, of the guidewire 5 relative to the handle 1 (e.g., advancing and retracting along the lumen). The guidewire management system 3020 can include an actuator 3022. The actuator 3022 can include a wheel or pair of wheels that can engage with the guidewire 5 (e.g., on either side). The actuator 3022 can advance and retract the guidewire 5 along the lumen by rotation while engaged with the guidewire 5. The actuator 3022 can advance and retract the guidewire 5 along an axis aligned with the lumen and/or the handle 1. The actuator 3022 can also lock the guidewire against movement relative to the lumen of the handle 1.

The guidewire management system 3020 can include a support 3026. The support 3026 can be coupled with the base 3006 or rail 3002. The support 3026 can be adjustable to align the guidewire 5 with the lumen of the handle 1. The support 3026 can include an actuatable joint 3027 for positioning the actuator 3022 and/or the guidewire 5.

In another embodiment, the actuator 3022 can include a linear actuator, electric motor, servo motor, or other mechanism for advancing and retracting. The actuator 3022 can include a servo controller that measures a position of the guidewire 5 along the axis relative to an initial position. The servo controller can provide positional feedback to a controller. The actuator 3022 can include a force sensor that measures a force applied to the guidewire 5 by the actuator 3022. The load sensor can provide force feedback to the controller. Optionally, the actuator 3022 can be integrated with the support 3026.

The actuator 3022 can include an engagement clamp 3024 or otherwise function to clamp onto the guidewire 5. The engagement clamp 3024 can releasably secure with the guidewire 5. The engagement clamp 3024 can be actuatable between a locked configuration and an unlocked configuration. The engagement clamp 3024 can be manually or automatically actuatable. The engagement clamp 3024 can include a passive fixing groove, pinching members or wheels, or other mechanism for securing the guidewire 5 thereto.

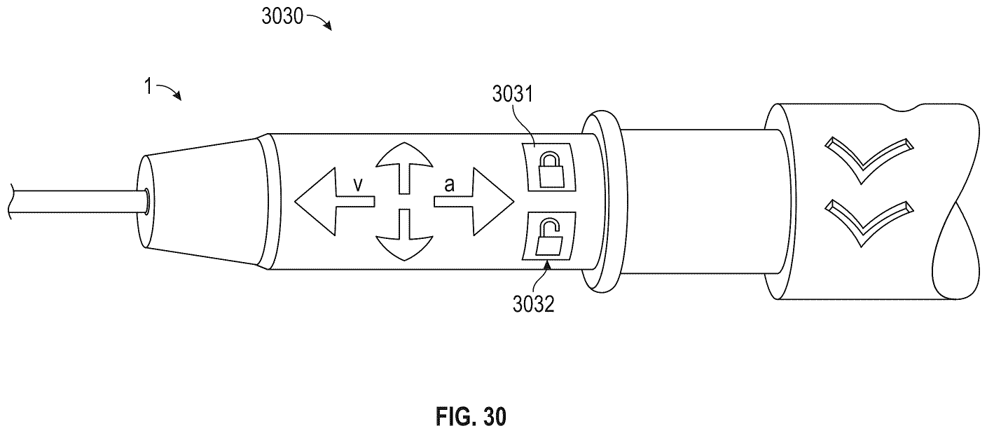

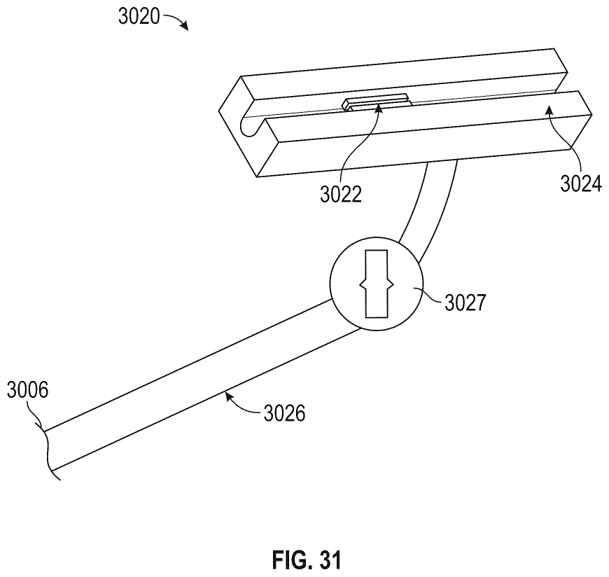

The guidewire management system 3020 can include a user interface 3030 (FIG. 30). The user interface can include one or more inputs such as buttons, dials, or the like to receive a user input. The controller can be programmed to move the guidewire 5 to selectably advance and retract along the lumen based on the user input. The controller can generate a motor control signal based on the user input signal and the actuator 3022 can receive the motor control signal and advance or retract the guidewire 5 along the axis based on the motor control signal. The motor control signal can be further based on a position of the guidewire 5 or on a force exerted on the guidewire 5 by the actuator 3022. The force can be detected by a sensor on the guidewire management system 3020, such as on the support 3026, actuator 3022, or clamp 3024.

The user interface 3030 can include fine and/or coarse controls for movement of the guidewire 5. The user interface can include a fine advance button V (ventricular direction), a fine retract button A (atrial direction), a coarse advance button a, and/or a coarse retract button v. The user interface can include a lock button 3031 and/or an unlock button 3032 for actuating the engagement clamp 3024 between the locked and unlocked configurations based on the user input signal. The guidewire management system 3020 can include a wireless (e.g., BLUETOOTH) communication system configured to transmit the user input signal from the user interface 3030 to the controller mounted on the support. The buttons may be substituted with other actuators (e.g., switches, levers, toggles, slides, etc.).

In some configurations, the guidewire management system 3020 can be configured to prevent or reduce the likelihood of tissue perforation by the guidewire. For example, the guidewire management system 3020 may include a strain gauge or force gauge incorporated into a slide assembly or grip interface that could provide a force feedback to the controller (e.g., one or more microprocessors). A force threshold may be programmed (in a manufacturing assembly stage and/or by an operator before or during a procedure) as a safeguard to ensure that forces are not generated that exceed a determined perforation force threshold. The programmed force threshold may advantageously be lower than the determined perforation force threshold.

The guidewire management system 3020 can be used with any delivery system and is not limited to use with the delivery systems or guidewires disclosed herein. The guidewire management system 3020 can be used to hold and/or control other guidewires or delivery system components (e.g., elongate tubes or elongate solid members).

The guidewire management system 3020 may also be capable of rotating or twisting the guidewire 5 or delivery system component. For example, the user interface 3030 may include one or more rotation buttons or inputs (e.g., clockwise input and counter-clockwise input). The guidewire management system 3020 may include one or more torque-limiting sensors that provide force feedback to the controller.

In some configurations, voltage or other input may be applied to the guidewire 5 to cause the guidewire to energize the guidewire to facilitate tissue ablation, cutting or other tissue modification and/or to change stiffness of at least a portion of the guidewire, as desired and/or required. For example, a coating or other portion of the guidewire at a proximal end of the guidewire outside the body may be energized (e.g., via a radiofrequency generator) so as to allow cutting of native valve leaflet tissue or to facilitate ablation of tissue to treat atrial fibrillation or other heart rhythm abnormalities. In some implementations, voltage may be applied to cause a phase change of at least a portion of the guidewire 5 (e.g., a guidewire formed at least partially of phase change nitinol or other material capable of changing shape due to phase changes) so as to generate heat to cause a change in a phase from shape memory phase to a superelastic phase or between other types of phases or configurations.

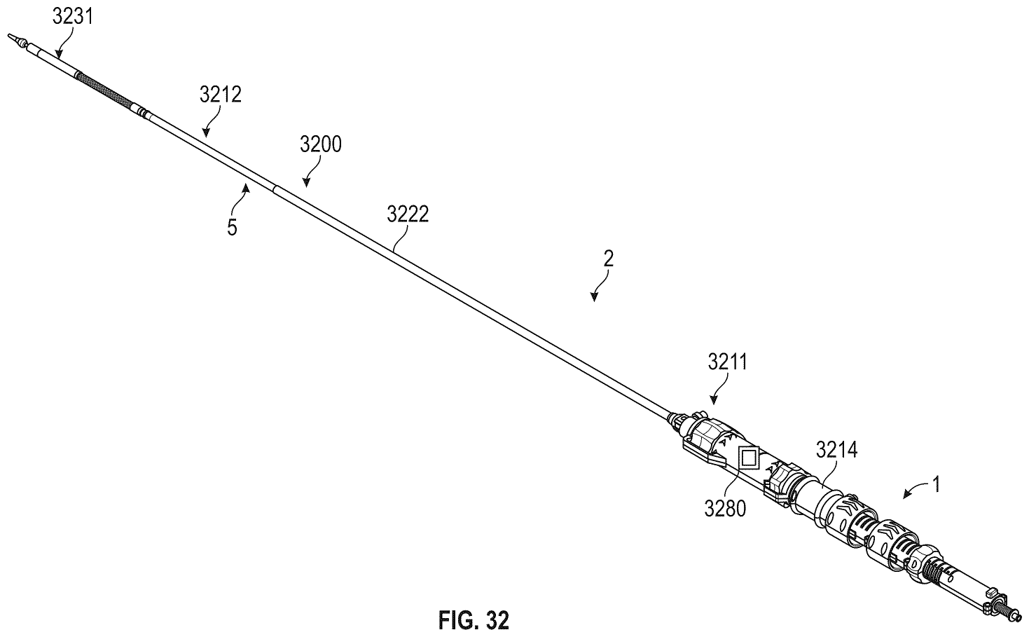

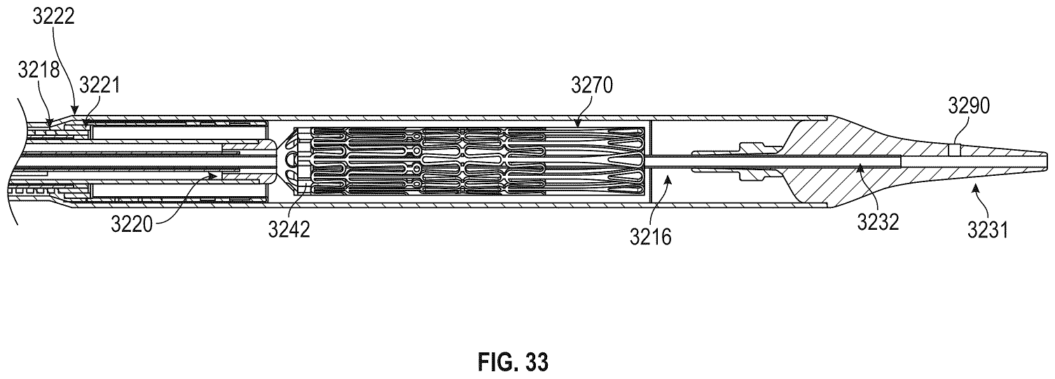

FIGS. 32-33 show a delivery system 3200 for delivering an expandable implant to a body location. Examples of delivery systems are described in detail in U.S. Pat. Pub. No. 2019/0008640, which is incorporated by reference herein. One aspect of the delivery system 3200 is the reduction of trauma to the heart. Trauma can occur due to excesses forces on the tissues of the body due to insertion and advancement of a distal end 3212 of the delivery system 3200 with a nose cone assembly 3231. Accordingly, the nose cone assembly 3231 can includes a force sensor 3290. The force sensor 3290 can be a strain gauge. The force sensor 3290 can be located between the proximal and distal ends of the nose cone. The force sensor 3290 can be embedded within the material of the nose cone or attached to an inner or outer side thereof.

The handle 3214 can include a haptic feedback device 3280 or other means for alarming a user of excessive forces detected by the force sensor 3290. The feedback device 3280 can include a vibrating motor, piezoelectric or other device. For example, the handle 3214 of the delivery system 3200 can vibrate if the nose cone assembly 3231 detects high forces (e.g., above a threshold force) when crossing the septum (mitral valve approach) or ventricular interactions in both tricuspid and mitral applications. Adding a strain gauge to the nose cone assembly 3231 can further detect forces on heart anatomy, especially interactions between the nose cone 3231 and ventricle. If forces are considered in excess of a predetermined threshold (e.g., may cause damage to heart or other tissue) the feedback device 3280 will vibrate a warning of the high forces (e.g., above a threshold force) to the user.

The delivery system 3200 can include an outer sheath assembly 3222 including an outer shaft having an outer lumen and a proximal end 3211 attached with the handle 3214 and a distal end 3212. As shown in FIG. 33, the outer sheath assembly 3222 can include an implant retention area 3216 that retains the expandable implant 3270 in a compressed configuration. A rail assembly 3220 can be located within the outer lumen. The rail assembly 3220 can include a rail shaft having a rail lumen and a proximal end and a distal end. The rail assembly 3220 can include one or more pull wires attached on an inner surface of the rail shaft and provide an axial force on the rail shaft to steer the rail assembly 3220. An inner assembly 3218 can be located within the outer lumen. The inner assembly 3218 can include an inner shaft having an inner lumen and a proximal end and a distal end. The inner assembly 3218 can include an inner retention member configured to be releasably attached to the expandable implant 3270. The outer sheath assembly 3222 and the inner assembly 3218 can move together distally relative to the rail assembly 3220 while the expandable implant 3270 remains in the compressed configuration. The outer sheath assembly 3222 can retract proximally relative to the inner assembly 3218 in order to at least partially expand the expandable implant 3270 from the compressed configuration. A mid shaft assembly 3221 can be within the outer lumen. The mid shaft assembly 3221 can include a mid-shaft having a middle lumen and a proximal end and a distal end. The mid shaft assembly 3221 can include an outer retention member 3242 configured to radially restrain at least a portion of the expandable implant 3270. A proximal end of the nose cone assembly 3231 can be located within the inner lumen. The nose cone assembly 3231 can include a nose cone shaft having a guide wire lumen 3232, a proximal and distal end, and a nose cone on the distal end. The mid shaft assembly 3221 and the nose cone assembly 3231 can move together distally with the outer sheath assembly and the inner assembly relative to the rail assembly 3220 while the expandable implant 3270 remains in the compressed configuration. The mid shaft assembly 3221 can retract proximally relative to the inner assembly 3218 in order to at least partially expand the expandable implant from the compressed configuration.

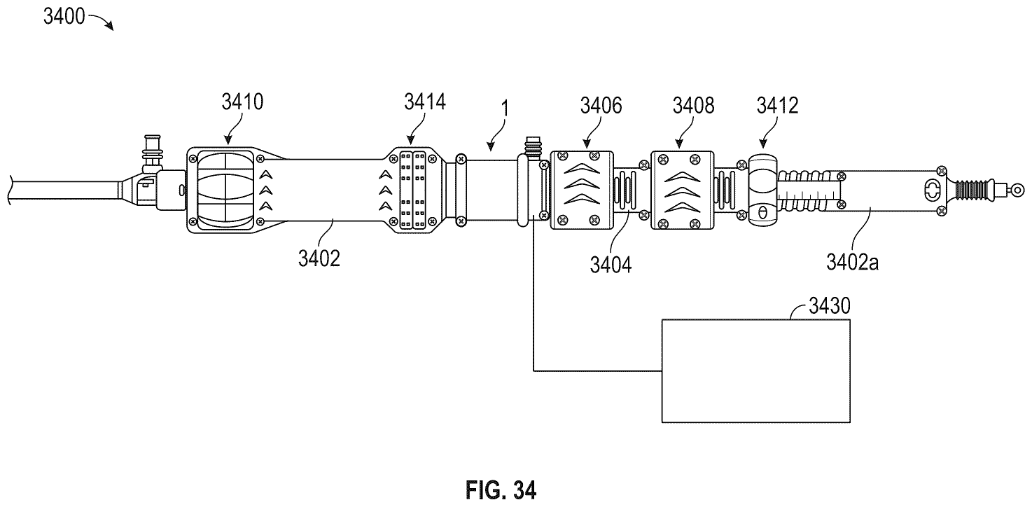

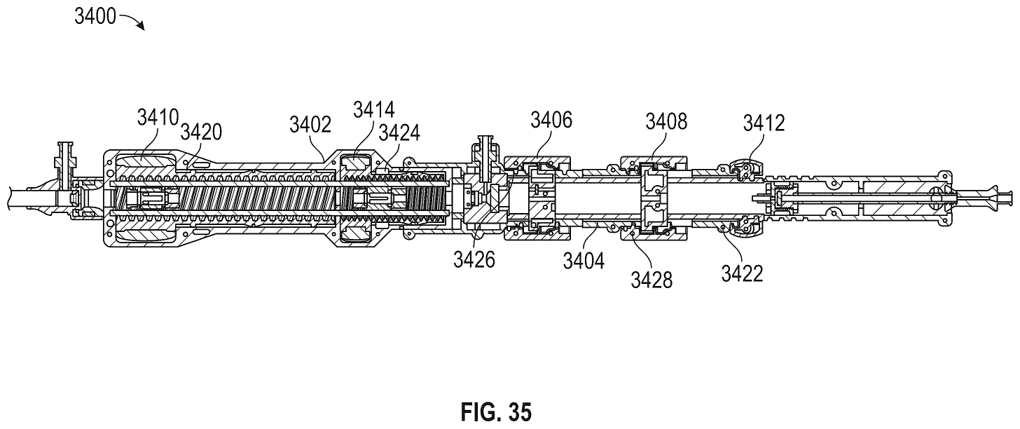

FIGS. 34-35 show a handle 3400 for a delivery system with electronic positional control knobs. The handle 3400 can be used as a part of a delivery system, such as the delivery system 3200 disclosed herein. The handle 3400 can include a delivery housing 3402 and a rail housing 3404. The delivery housing 3402 can be movable relative to the rail housing 3404.

The positional control knobs can include rotatable actuators. The position or status of each the rotatable actuators can be measured with one or more encoders. The encoders can allow for accurate measurement of travel for the lumens, flex members, and/or depth adjusters. The encoders can include any encoder type including magnetic, optical, inductive, capacitive, resistive, or mechanical. The handle 3400 can include or be electronically connected with a processor (not shown) configured to receive a signal from each of the encoders and output a positional status of each of the actuators. In some embodiments, the one or more encoders are adapted or configured for measuring the travel (axial or rotational) of lumens, flex members, and/or depth adjusters and can be mechanical encoders. Such mechanical encoders can have detents that can indicate rotational positions corresponding to different translational or depth positions of the above-mentioned mechanisms of the handle 3400. The detents may provide tactile and/or audible clicks when engaged at specific rotational positions. For example, the detents (or other physical components) can reduce in spacing as a function of an amount of rotation (e.g., progressing from 2 mm to 0.5 mm spacings) so as to create a sensation of reaching a point of furthest flex for each control (e.g., control knob). A user interface 3430 can display the positional status of each of the rotatable actuators. The user interface 3430 can include a digital screen used during surgery. The positional status can be displayed in relation to a model of a patient's body. The user interface 3430 can provide the limits of the movements of each of the control knobs. This can assist in planning of a procedure based on the available movement of the delivery system. A warning can also be displayed based on nearing control knob/movement limits.

The rotatable actuators can include a sheath knob 3410, a mid shaft knob 3414, a distal rail flex knob 3406, a proximal rail flex knob 3408, and/or a depth knob 3412. The rail housing 3404 can include the distal rail flex knob 3406. The rotation of the distal rail flex knob 3406 can provide an axial force on a first pull wire connected with a rail through an adapter connected thereto. The distal rail flex knob 3406 can be mounted about a circumference of the rail housing 3404. The distal rail flex knob 3406 can rotate about a longitudinal axis of the rail housing 3404. A first encoder 3426 can be mounted on the rail housing 3404 to measure a position of the knob 3406. A code track can be printed on the knob 3406, such as along a side thereof.

The rail housing 3404 can include the proximal rail flex knob 3408. The rotation of the knob 3408 can provide an axial force on a second pull wire connected with a rail through an adapter connected thereto. The proximal rail flex knob 3408 can be mounted about a circumference of the rail housing 3404. The proximal rail flex knob 3408 can rotate about a longitudinal axis of the rail housing 3404. A second encoder 3428 can be mounted on the rail housing 3404 to measure a position of the knob 3408. A code track can be printed on the knob 3408.

The delivery housing 3402 can include the sheath knob 3410 and the mid shaft knob 3414. The sheath knob 3410 can have an outer sheath assembly distally relative to the delivery housing 3402. The mid shaft knob 3414 can retract proximally a mid shaft assembly relative to the delivery housing 3402. The sheath knob 3410 and the mid shaft knob 3414 can be mounted about a circumference of the delivery housing 3402 and rotate about a longitudinal axis of the delivery housing 3402. A third encoder 3420 can be mounted on the delivery housing 3402 to measure a position of the knob 3410. A code track can be printed on the knob 3410. A fourth encoder 3424 can be mounted on the delivery housing 3402 to measure a position of the knob 3414. A code track can be printed on the knob 3414.

The depth knob 3412 can move the delivery housing 3402a relative to the rail housing 3404. An internal thread of the depth knob 3412 can engage with external threads of the delivery housing 3402a to adjust a position thereof along the longitudinal axis relative to the rail housing 3404. A fifth encoder 3422 can be mounted on the rail housing 3404 and measure a position of the depth knob 3412, which corresponds to a position of the delivery housing 3402 relative to the rail housing 3404.

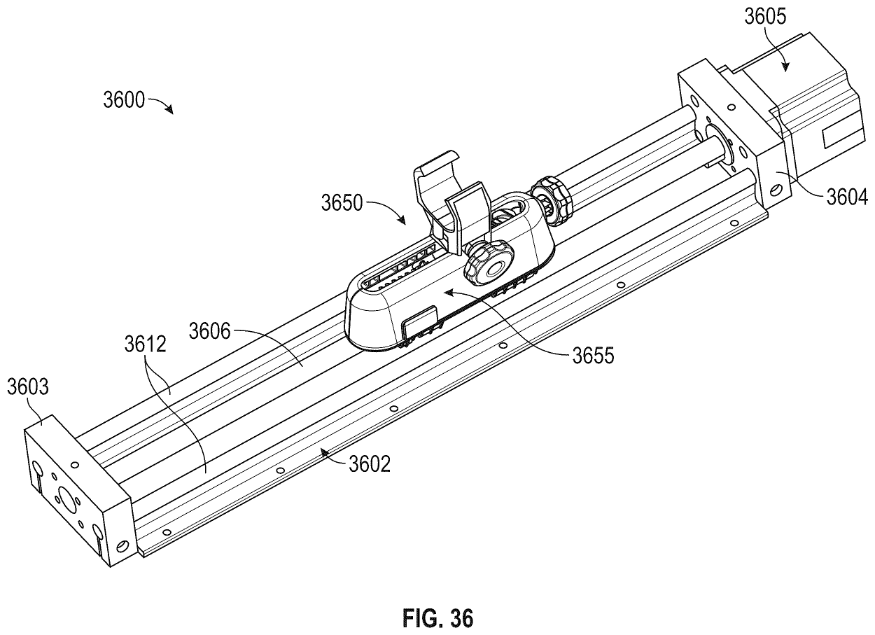

FIG. 36 illustrates another embodiment of a stabilizer 3600. The stabilizer 3600 can include a motorized rail system 3602. The motorized rail system 3602 can be mountable on a base, such as the base 1006 of stabilizer 1000 shown in FIGS. 1A-B. The motorized rail system 3602 can include a pair of rail members 3612. The rail members 3612 can extend between a first end plate 3603 and a second end plate 3604. The motorized rail system 3602 can include a motor 3605. The motor 3605 can be mounted on the second end plate. The motor 3605 can include an electric motor, servo motor, stepper motor or another type of motor. A threaded shaft 3606 can be coupled with the motor 3605. The threaded shaft 3606 can extend between the first and second rail member and be supported by the first and second end plates (e.g., journaled on bearings).

The motorized rail system 3602 can include a support 3650. The support 3650 can receive and/or clamp about a portion of a handle of a delivery system. The support 3650 can include any of the features and structures of the supports disclosed herein (e.g., supports 1150, 1320, 1340, 1350, 1900, 2000, 2100, 2200). The support 3650 can be operable between an open configuration and a closed configuration (e.g., locked) position for receiving the handle therein. The support 3650 can be mounted on one or both of the rail members and slidable between the first and second end plates 3603 and 3604 (e.g., along a longitudinal axis of the rail system). The support 3650 can include a threaded carriage 3655. The threaded carriage 3655 can be configurable between an engaged position that is engaged with the threaded shaft 3606 and a disengaged position away from the threaded shaft 3606. In the disengaged position, the support 3650 can be freely slid along the rail members in first and second directions along the longitudinal axis.

The motor 3605 can be coupled with a controller for actuating the motor based on a motor control signal. The motor control signal can be generated based on an input from a user through a user interface. Actuating the motor 3605 can spin the threaded shaft 3606. In the engaged position of the threaded carriage, the spinning of the threaded shaft 3606 can move the support 3650 along the rail members in the first or second directions along the longitudinal axis. The support 3650 can be moved in discrete increments. A precise location of the support 3650 can be measured by the rail system 3602. The rail system 3602 can include one or more encoders that track a position of the support 3650 based on a position of the threaded shaft 3606 and a starting position of the support 3650. The controller can be remotely controlled and/or automatically controlled.

Optionally, the motorized rail system 3602 can include a second support (not shown). The second support can be a hub nest or passive support, such as the hub nests and passive support disclosed above herein. The second support can be mounted on the rail members. The second support can be coupled with the support 3650 and move therewith. Alternatively, the second support can be movable independently from the support 3650. In one example, the second support can be engaged with a second threaded actuator. In another alternative, the second support can be configured to move independently from the support 3650 or moved therewith, such as through a threaded carriage that is configurable between an engaged position and a disengaged position relative to the threaded shaft.

### Certain Terminology

Terms of orientation used herein, such as “upper,” “lower,” “top,” “bottom,” “proximal,” “distal,” “longitudinal,” “lateral,” and “end,” are used in the context of the illustrated example. However, the present disclosure should not be limited to the illustrated orientation. Indeed, other orientations are possible and are within the scope of this disclosure. Terms relating to circular shapes as used herein, such as diameter or radius, should be understood not to require perfect circular structures, but rather should be applied to any suitable structure with a cross-sectional region that can be measured from side-to-side. Terms relating to shapes generally, such as “circular,” “cylindrical,” “semi-circular,” or “semi-cylindrical” or any related or similar terms, are not required to conform strictly to the mathematical definitions of circles or cylinders or other structures, but can encompass structures that are reasonably close approximations.

Conditional language, such as “can,” “could,” “might,” or “may,” unless specifically stated otherwise, or otherwise understood within the context as used, is generally intended to convey that certain examples include or do not include, certain features, elements, and/or steps. Thus, such conditional language is not generally intended to imply that features, elements, and/or steps are in any way required for one or more examples.

Conjunctive language, such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

The terms “approximately,” “about,” and “substantially” as used herein represent an amount close to the stated amount that still performs a desired function or achieves a desired result. For example, in some examples, as the context may dictate, the terms “approximately,” “about,” and “substantially,” may refer to an amount that is within less than or equal to 10% of the stated amount. The term “generally” as used herein represents a value, amount, or characteristic that predominantly includes or tends toward a particular value, amount, or characteristic. As an example, in certain examples, as the context may dictate, the term “generally parallel” can refer to something that departs from exactly parallel by less than or equal to 20 degrees. All ranges are inclusive of endpoints.

### Summary

Several illustrative examples of stabilizer systems and related delivery system improvements have been disclosed. Although this disclosure has been described in terms of certain illustrative examples and uses, other examples and other uses, including examples and uses which do not provide all of the features and advantages set forth herein, are also within the scope of this disclosure. Components, elements, features, acts, or steps can be arranged or performed differently than described and components, elements, features, acts, or steps can be combined, merged, added, or left out in various examples. All possible combinations and sub-combinations of elements and components described herein are intended to be included in this disclosure. No single feature or group of features is necessary or indispensable.

Certain features that are described in this disclosure in the context of separate implementations can also be implemented in combination in a single implementation. Conversely, various features that are described in the context of a single implementation also can be implemented in multiple implementations separately or in any suitable subcombination. Moreover, although features may be described above as acting in certain combinations, one or more features from a claimed combination can in some cases be excised from the combination, and the combination may be claimed as a subcombination or variation of a subcombination.

Any portion of any of the steps, processes, structures, and/or devices disclosed or illustrated in one example in this disclosure can be combined or used with (or instead of) any other portion of any of the steps, processes, structures, and/or devices disclosed or illustrated in a different example or flowchart. The examples described herein are not intended to be discrete and separate from each other. Combinations, variations, and some implementations of the disclosed features are within the scope of this disclosure.

While operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Additionally, the operations may be rearranged or reordered in some implementations. Also, the separation of various components in the implementations described above should not be understood as requiring such separation in all implementations, and it should be understood that the described components and systems can generally be integrated together in a single product or packaged into multiple products. Additionally, some implementations are within the scope of this disclosure.

Further, while illustrative examples have been described, any examples having equivalent elements, modifications, omissions, and/or combinations are also within the scope of this disclosure. Moreover, although certain aspects, advantages, and novel features are described herein, not necessarily all such advantages may be achieved in accordance with any particular example. For example, some examples within the scope of this disclosure achieve one advantage, or a group of advantages, as taught herein without necessarily achieving other advantages taught or suggested herein. Further, some examples may achieve different advantages than those taught or suggested herein.

Some examples have been described in connection with the accompanying drawings. The figures are drawn and/or shown to scale, but such scale should not be limiting, since dimensions and proportions other than what are shown are contemplated and are within the scope of the disclosed invention. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various examples can be used in all other examples set forth herein. Additionally, any methods described herein may be practiced using any device suitable for performing the recited steps.

For purposes of summarizing the disclosure, certain aspects, advantages and features of the inventions have been described herein. Not all, or any such advantages are necessarily achieved in accordance with any particular example of the inventions disclosed herein. No aspects of this disclosure are essential or indispensable. In many examples, the devices, systems, and methods may be configured differently than illustrated in the figures or description herein. For example, various functionalities provided by the illustrated modules can be combined, rearranged, added, or deleted. In some implementations, additional or different processors or modules may perform some or all of the functionalities described with reference to the examples described and illustrated in the figures. Many implementation variations are possible. Any of the features, structures, steps, or processes disclosed in this specification can be included in any example.

In summary, various examples of stabilizer systems and related delivery system improvements and related methods have been disclosed. This disclosure extends beyond the specifically disclosed examples to other alternative examples and/or other uses of the examples, as well as to certain modifications and equivalents thereof. Moreover, this disclosure expressly contemplates that various features and aspects of the disclosed examples can be combined with, or substituted for, one another. Accordingly, the scope of this disclosure should not be limited by the particular disclosed examples described above, but should be determined only by a fair reading of the claims.

What is claimed is:

1. A universal stabilizer adapted or configured for use with multiple different delivery systems, the universal stabilizer comprising:
a rail extending along a longitudinal direction and having a first end, a second end, an upper facing surface, a lower facing surface, and first and second sides extending between the first and second ends; and
a rail dock mounted on the upper facing surface of the rail, the rail dock comprising:
first and second channel members spaced apart to receive the first and second sides of the rail therebetween, the first and second channel members including distal ends that overhang the lower facing surface of the rail and prevent removal of the rail dock from the rail in a vertical direction; and
a brake assembly configured to be actuated between a first configuration in which the rail dock is configured to translate along the rail and a second configuration in which the rail dock is prevented from translating along the rail,
wherein the brake assembly includes a toggle member with a first button on a first side and a second button on a second side, wherein pressing the first button actuates the brake assembly from the first configuration to the second configuration and pressing the second button shifts the brake assembly from the second configuration to the first configuration.

2. The stabilizer of claim 1, further comprising a guidewire management system operably coupled to the rail or a base to which the rail is attached, wherein the guidewire management system is configured to support a guidewire over which a delivery system is configured to be advanced.

3. The stabilizer of claim 1, wherein the rail dock includes a first support for a handle of the delivery system.

4. The stabilizer of claim 3, wherein the rail dock includes a carriage oriented along the longitudinal direction, the first support mounted on the carriage and movable along the longitudinal direction relative to the rail dock.

5. The stabilizer of claim 4, wherein the carriage is threadedly mounted on a travel screw, the travel screw connected with a twist knob for moving the carriage.

6. The stabilizer of claim 3, wherein the first support includes a fixed member and a movable member movably coupled with the fixed member, the first support actuatable between an open configuration and a closed configuration.

7. The stabilizer of claim 6, wherein the first support is actuatable between the open configuration and the closed configuration by a rotation of a knob, the rotation being less than about 180°.

8. The stabilizer of claim 6, further comprising a lock switch, the lock switch rotatable between a locked position that holds the first support in the closed configuration, and an unlocked position in which the first support is allowed to move between the open and closed configurations.

9. The stabilizer of claim 3, wherein the first support comprises a worm gear connected with a knob and a worm wheel connected with the handle, and rotation of the knob controls rotation of the handle about a longitudinal axis.

10. The stabilizer of claim 3, wherein the first support comprises an elastic strap with at first end secured to a first side of the first support, a middle section extending over the handle, and a second side securable to a second side of the first support.

11. A surgical system comprising the stabilizer of claim 3, further comprising:
a base to which the rail of the stabilizer is configured to attach to; and the delivery system comprising the handle with which the first support of the stabilizer is configured to engage, the delivery system being configured to deliver a heart valve prosthesis to replace a native heart valve.

12. A universal stabilizer adapted or configured for use with multiple different delivery systems, the universal stabilizer comprising:
a rail extending along a longitudinal direction and having a first end, a second end, an upper facing surface, a lower facing surface, and first and second sides extending between the first and second ends; and
a rail dock mounted on the upper facing surface of the rail, the rail dock comprising:
first and second channel members spaced apart to receive the first and second sides of the rail therebetween, the first and second channel members including distal ends that overhang the lower facing surface of the rail and prevent removal of the rail dock from the rail in a vertical direction; and
a brake assembly configured to be actuated between a first configuration in which the rail dock is configured to translate along the rail and a second configuration in which the rail dock is prevented from translating along the rail,
wherein the brake assembly includes a toggle member with at least one push button on a first side of the brake assembly, wherein pressing the at least one push button actuates the brake assembly from the first configuration to the second configuration and releasing the at least one button actuating the brake assembly from the second configuration to the first configuration.

13. The stabilizer of claim 12, wherein the brake assembly includes a brake member that engages and disengages from the upper facing surface of the rail, and wherein the brake assembly includes a ramp connected with the toggle member, the ramp configured to actuate the brake member to engage and disengage from the upper facing surface of the rail.

14. The stabilizer of claim 12, wherein the distal ends of the channel members are closer together than a width of the rail and fixed in position, such that the rail dock is loaded onto the rail by alignment of the first and second channels with the first and second sides at the first end of the rail and cannot be removed in a vertical direction.

15. The stabilizer of claim 12, further comprising a second support coupled with the rail, wherein the second support is a hub nest configured to receive an introducer hub or sheath hub associated with a delivery system, the hub nest including a locking post that inserts within a receiving aperture in the upper facing surface of the rail and rotates to removably lock into place on the rail.

16. A universal stabilizer adapted or configured for use with multiple different delivery systems, the universal stabilizer comprising:
a rail extending along a longitudinal direction and having a first end, a second end, an upper facing surface, a lower facing surface, and first and second sides extending between the first and second ends; and
a rail dock mounted on the upper facing surface of the rail, the rail dock comprising:
a first channel member on a first side of the rail dock aligned with the first side of the rail;
a second channel member on a second side of the rail dock, the second channel member on a plate biased inwardly towards the second side of the rail; and
a button that couples with the plate and pressing the button shifts the plate and second channel member away from the rail to permit the rail dock to translate along the rail and releasing the button shifts the plate and the second channel member into the second side of the rail to prevent the rail dock from translating along the rail.

17. The stabilizer of claim 16, wherein the first and second channel members each include a projection with a distal end that overhangs the lower facing surface of the rail.

18. The stabilizer of claim 16, wherein the rail dock includes a lockable support or a passive handle support.

19. The stabilizer of claim 16, further comprising:
a third channel member biased into engagement with the either the first or second sides of the rail, wherein the first side of the rail includes a textured surface.

20. The stabilizer of claim 16, further comprising a blocking member actuatable between a secured position in which the rail dock is prevented from being removed from the rail in a vertical direction and an unsecured position in which the rail dock is permitted to be removed from the rail in the vertical direction.
